(19) United States
(12) Patent Application Publication
(10) Pub. No.: US 2026/0295013 A1
(43) Pub. Date: Oct. 1, 2026

(54) COMBINATION TREATMENTS TO PROMOTE EPITHELIAL INTEGRITY AND TREAT OCULAR DISORDERS

(71) Applicant: WAYNE STATE UNIVERSITY, Detroit, MI (US)

(72) Inventors: Elizabeth Berger, Detroit, MI (US); Gabriel Sosne, Detroit, MI (US)

(73) Assignee: WAYNE STATE UNIVERSITY, Detroit, MI (US)

(21) Appl. No.: 19/483,112

(22) PCT Filed: May 10, 2024
(86) PCT No.: PCT/US2024/028810

§ 371 Date: Nov. 10, 2025

Related U.S. Application Data
(60) Provisional application No. 63/501,634, filed on May 11, 2023.

(51) Int. Cl. A61K 38/22; A61K 45/06; A61P 17/02; A61P 27/02

(52) U.S. Cl. CPC A61K 38/2292; A61K 38/2278; A61K 45/06; A61P 17/02; A61P 27/02

(57) ABSTRACT

Combination treatments to promote epithelial integrity and to treat ocular disorders, such as keratoepitheliopathy, impaired corneal wound healing, and corneal neuropathy, are described. The combination treatments include thymosin beta 4 (Tb4) and vasoactive intestinal peptide (VIP), or prodrugs or active fragments thereof. In various embodiments, the combination treatments can be used promote epithelial integrity and to treat ocular disorders in diabetic subjects.

### CROSS-REFERENCE TO RELATED APPLICATION

This application is a U.S. National Phase application based on International Patent Application No. PCT/US2024/028810, filed on May 10, 2024, which claims priority to U.S. Provisional Patent Application No. 63/501,634 filed on May 11, 2023, the contents of both of which are incorporated by reference herein in their entirety.

### STATEMENT REGARDING FEDERALLY SPONSORED RESEARCH OR DEVELOPMENT

This invention was made with government support under EY029836 awarded by the National Eye Institute. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3IR6606.XML. The file is 6, 198 bytes 5,999 bytes, was created on Nov. 6, 2025, and is being submitted electronically via Patent Center.

### FIELD OF THE DISCLOSURE

The present disclosure describes combination treatments to promote epithelial integrity and to treat ocular disorders, such as keratoepitheliopathy, impaired corneal wound healing, and corneal neuropathy. The combination treatments include thymosin beta 4 (Tb4) and vasoactive intestinal peptide (VIP), or prodrugs or active fragments thereof. In various embodiments, the combination treatments can be used to promote epithelial integrity and to treat ocular disorders in diabetic subjects.

### BACKGROUND OF THE DISCLOSURE

Through the course of day-to-day life, individuals are exposed to numerous external and internal insults that can compromise epithelial integrity. For example, external insults can pierce or lacerate the skin, compromising epithelial integrity. Moreover, 425 million people are diagnosed with diabetes, which is the leading cause of adult-onset blindness. Diabetes-induced corneal complications include: keratoepitheliopathy, impaired corneal wound healing and corneal neuropathy. Despite the prevalence of diabetic retinopathy, 70% of adult diabetic patients develop visually debilitating corneal complications, including impaired wound healing. Unfortunately, treatment for diabetes-induced corneal damage remains limited. Currently available treatments include aldose reductase inhibitors and strict glycemic control.

### SUMMARY OF THE DISCLOSURE

The present disclosure describes combination treatments to promote epithelial integrity and to treat ocular disorders, such as keratoepitheliopathy, impaired corneal wound healing, and corneal neuropathy. The combination treatments include thymosin beta 4 (Tb4) and vasoactive intestinal peptide (VIP), or prodrugs or active fragments thereof. In various embodiments, the combination treatments can be used to promote epithelial integrity and treat ocular disorders in diabetic subjects.

### BRIEF DESCRIPTION OF THE FIGURES

FIGS. 1A-1E. Barrier function of HUCLs reflected by real-time bioimpedance analysis using an AC frequency scan. HUCLs were seeded (60,000 cells/well) on a 96W20idf ECIS array. Three-dimensional representation of the log-normalized impedance (y-axis) as a function of the log frequency of both AC (x-axis) and time (z-axis). Cells maintained in NG, HG, HG+Tβ4/VIP (FIGS. 1A-1C), HG+Tβ4, and HG+VIP (FIGS. 1D, 1E) are represented. Time=0 denotes time of inoculation. Log-normalized impedance values are also represented by the color bar.

FIGS. 2A-2I. Real-time monitoring of HUCL impedance (measured at an AC frequency of 32 kHz) (2A), resistance (measured at an AC frequency of 4000 Hz) (2B), and capacitance (measured at an AC frequency of 64 kHz) (2C) on a 96W20idf ECIS array. Cells at a 60,000 seeding density were maintained in NG, HG, HG+Tβ4/VIP, HG+Tβ4, and HG+VIP. Impedance, resistance, and capacitance were measured from the time of inoculation (T=0 h) to 120 h after treatment application. Bar graph representation of normalized impedance (2D, 2G), resistance (2E, 2H), and capacitance (2F, 2I) endpoint values and area under the curve, respectively, for each group. Data shown are mean±SEM; n=5/group. *p≤0.05; **p≤0.01; ***p≤0.001.

FIGS. 3A-3F. Mathematical modeling of Rb, a, and Cm for HUCLs maintained in NG, HG, HG+Tβ4/VIP, HG+Tβ4, and HG+VIP. Modeled normalized parameters Rb (3A), a (3B), and Cm (3C) were traced over 120 hr. Bar graphs represent total and endpoint values as calculated for Rb (3D), a (3E), and Cm (3F). Data shown are the mean±SD; n=5/group. *p≤0.05; **p≤ 0.01; ***p≤0.001.

FIGS. 4A-4E. Cell migration/electroporation wounding of HUCLs monitored by real-time bioimpedance analysis using an AC frequency scan. HUCLs were seeded (60,000 cells/well) on a 96W1+ECIS array. 3D representation of the log-normalized impedance (x-axis) as a function of the log frequency of both AC (y-axis) and time (z-axis). Cells maintained in NG (4A), HG (4B), HG+Tβ4/VIP (4C), HG+Tβ4 (4D), and HG+VIP (4E) are represented. Wound induction is denoted by the red arrow. Dark red peaks reflect a media change. Time=0 denotes time of inoculation. Log-normalized impedance values are also represented by the color bar.

FIGS. 5A-5G. Real-time monitoring of HUCL impedance (measured at 32 kHz) (5A), resistance (measured at 4000 Hz) (5B), and capacitance (measured at 64 kHz) (5C) on a 96W1+ECIS array. Cells at a 60,000 seeding density were maintained in NG, HG, HG+Tβ4/VIP, HG+Tβ4, and HG+VIP. Impedance, resistance, and capacitance were measured from the time of wounding (T=30 h) to 70 h post-wound. Bar graphs represent total values as calculated for normalized impedance (5D), resistance (5E), capacitance (5F), and cell velocity (μm/h) (5G), presented for each group. Data shown are the mean±SEM; n=5/group. *p≤0.05; **p≤0.01; ***p≤0.001.

FIGS. 6A-6D. Detection of select tight junction complex components as detected by Western blot in HUCLs maintained in NG, HG, HG+Tβ4/VIP, HG+Tβ4, and HG+VIP. Protein levels for ZO-1 (6A), occludin (6B), claudlin-1 (6C), and ZO-2 (6D) are presented as a ratio to β-actin±SD. n=5/group. *p≤0.05; **p≤0.01.

FIG. 7. Tight junction staining in HUCLs with DAPI nuclear stain. Positive immunostaining of ZO-1 (top row) and occludin (middle row) is shown in green, highlighting the localization of tight junctions. DAPI staining (blue) provides visualization of the cellular nuclei. Ki67 staining (green) (bottom row) indicates the presence of the Ki-67 protein within the nucleus of cells undergoing active proliferation. Magnification=40×. Scale bar=20 μm.

FIGS. 8A-8C. Merged images from FIG. 7 are provided herein as single channel images, illustrating the localization of tight junction proteins, ZO-1 (8A) and occludin (mid8Bdle), along with Ki-67 (8C) as a marker of cellular proliferation. DAPI nuclear stain is shown in blue. Magnification=40×.

### DETAILED DESCRIPTION

Through the course of day-to-day life, individuals are exposed to numerous external and internal insults that can comprise epithelial integrity. For example, external insults can pierce or lacerate the skin, compromising epithelial integrity. Moreover, 425 million people are diagnosed with diabetes, which is the leading cause of adult-onset blindness. Diabetes-induced corneal complications include: keratoepitheliopathy, impaired corneal wound healing and corneal neuropathy. Despite the prevalence of diabetic retinopathy, 70% of adult diabetic patients develop visually debilitating corneal complications, including impaired wound healing. Unfortunately, treatment for diabetes-induced corneal damage remains limited. Currently available treatments include aldose reductase inhibitors and strict glycemic control.

The present disclosure describes combination treatments to promote epithelial integrity and to treat ocular disorders, such as keratoepitheliopathy, impaired corneal wound healing, and corneal neuropathy. The combination treatments include thymosin beta 4 (Tb4) and vasoactive intestinal peptide (VIP), or prodrugs or active fragments thereof. In various embodiments, the combination treatments can be used to promote epithelial integrity and treat ocular disorders in diabetic subjects.

Aspects of the disclosure are now described with additional details and options as follows: (i) Tβ4 and VIP; (ii) Compositions; (iii) Alternative Up-Regulation Methods; (iv) Methods of Use; (v) Exemplary Embodiments; (vi) Experimental Examples; and (vii) Closing Paragraphs.

(i) Tβ4 and VIP. Tβ4 is a small, naturally occurring 43 amino-acid protein that promotes wound healing and reduces corneal inflammation [Goldstein et al., Expert Opin. Boil. Ther. 2012, 12, 37-51.]. It is highly conserved across species and is expressed in all tissues and cell types except red blood cells [Sosne & Kleinman, Investig. Ophthalmol. Vis. Sci. 2015, 56, 5110-5117]. Tβ4 possesses regenerative properties, including full-thickness dermal wound repair and modulation of wound site inflammation [Qiu, et al., FASEB J. 2011, 25, 1815-1826]. It has also been shown to reduce non-healing epithelial defects in diabetic patients with neurotrophic keratopathy.

The sequence of Tb4 is: ACSDKPDMAEIEKFDKSKLKKTETQEKNPLPSKETIEQEKQAHIDNEMEEEAVDGSNGERGARA EANKNAGPRTTGAPRPAKSA (SEQ ID NO: 1). Exemplary active fragments of Tb4 include: 1) Ac-SDKP (N-Acetyl-SDKP) (SEQ ID NO: 2)—this is a tetrapeptide that is produced by the cleavage of proTb4 at the N-terminus . . . 2) Tb10 (SDKP) (SEQ ID NO: 3)—this is a tetrapeptide that is produced by the cleavage of proTb4 at the C-terminus. Tb10 is similar to Tb4 in its biological activity. 3) Tb6 (KPGP) (SEQ ID NO: 4)—this is a tripeptide that is produced by the cleavage of Tb10. Tb9 is also similar to Tb4 in its biological activity.

One of the most common isoforms of Tb4 in humans is Tb4X, which is produced by alternative splicing of the TMSB4X gene. Tb4X is identical to the canonical Tb4 sequence except for deletion of three amino acids (GGN) near the N-terminus.

Tβ4 is synthesized as a larger precursor molecule prothymosin beta-4 (proTb4), which is cleaved by specific enzymes to produce the active fragments of Tb4 along with other active fragments such as Ac-SDKP and Tb10.

VIP is a widely distributed neuropeptide that functions as an immunomodulator of the host response, not exclusively anti-inflammatory. One recent publication demonstrated that VIP treatment of HRECs significantly reduced HG-induced TNF-α and VEGF levels while increasing the expression of pro-resolving lipid mediator RvD1 and its receptor GPR32.

VIP is produced as a larger precursor molecule that undergoes proteolytic processing to produce several active fragments. The sequence of VIP, its active fragments, and precursors are: 1. PreproVIP: this is the precursor protein of VIP that contains 170 amino acids in humans. It consists of a signal peptide followed by VIP and related peptides, including peptide histidine isoleucine (PHI), peptide histidine valine (PHV), and secretin. 2. ProVIP: this is the intermediate precursor of VIP that is produced by the removal of the signal peptide from PreproVIP. ProVIP consists of 122 amino acids in humans. 3. VIP: the mature form of VIP is a 28 amino acid peptide that is produced by the proteolytic cleavage of ProVIP. The sequence of VIP is: HSDAVFTDNYTRLRKQMAVKKYLNSILN-NH2 SEQ ID NO: 5). 4. N-terminal VIP fragment (1-12): this is a 12 amino acid fragment of VIP that is produced by the cleavage of VIP at its N-terminus. This fragment is biologically active and has been shown to have similar effects to VIP in various physiological processes. 5. C-terminal VIP fragment (22-28): this is a 7 amino acid fragment of VIP that is produced by the cleavage of VIP at its C-terminus. This fragment is also biologically active and has been shown to have similar effects to VIP in various physiological processes.

In certain examples, Tβ4 and VIP can be up-regulated by administering prodrugs of Tβ4 and VIP or prodrugs of Tβ4 and VIP variants. Prodrugs refer to a protein that can undergo biotransformation (e.g., either spontaneous or enzymatic) within a subject to release, or to convert to, (e.g., enzymatically, mechanically, electromagnetically, etc.) an active or more active form of a protein. Prodrugs can be used to overcome issues associated with stability, toxicity, lack of specificity, or limited bioavailability. Some preferred prodrugs are variants of proteins that have sequences that are cleavable under metabolic conditions. Exemplary prodrugs become active or more active in vivo when they undergo a biochemical transformation (e.g., phosphorylation, hydrogenation, dehydrogenation, glycosylation, etc.). See e.g., Bundgard, Design of Prodrugs, pp. 7-9, 21-24, Elsevier, Amsterdam (1985); and Silverman, The Organic Chemistry of Drug Design and Drag Action, pp. 352-401, Academic Press, San Diego, CA (1992)).

“Variants” include proteins having one or more amino acid additions, deletions, stop positions, or substitutions, as compared to a reference protein disclosed herein. SEQ ID NO: 1 represents the amino acid sequence of human Tβ4 and is a reference sequence for purposes of the present disclosure. SEQ ID NO: 2 represents the amino acid sequence of VIP and is a reference sequence for purposes of the present disclosure.

An amino acid substitution can be a conservative or a non-conservative substitution.

Variants of proteins disclosed herein can include those having one or more conservative amino acid substitutions. A “conservative substitution” or “conservative amino acid substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other groups including amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Variants of proteins disclosed herein also include proteins with at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to a protein sequence disclosed herein.

Variants of therapeutic proteins disclosed herein include proteins that share: 70% sequence identity with SEQ ID NO: 1, 2, 3, 4, or 5, Tb4X, proTb4, PreproVIP, ProVIP, N-terminal VIP fragment (1-12), or C-terminal VIP fragment (22-28); 75% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 80% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 81% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 82% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 83% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 84% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 85% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 86% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 87% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 88% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 89% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 90% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 91% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 92% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 93% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 94% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 95% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 96% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 97% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); 98% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28); or 99% sequence identity with any of SEQ ID NO: SEQ ID NO: 1, 2, 3, 4, or 5, Tb4X, proTb4, PreproVIP, ProVIP, N-terminal VIP fragment (1-12), or C-terminal VIP fragment (22-28).

“% sequence identity” refers to a relationship between two or more sequences, as determined by comparing the sequences. In the art, “identity” also means the degree of sequence relatedness between protein or nucleotide sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.
(ii) Compositions. Compositions disclosed herein include one or more proteins disclosed herein with a pharmaceutically acceptable carrier.

Particular embodiments of compositions disclosed herein are formulated for systemic administration. In particular embodiments, the compositions disclosed herein can be formulated for topical administration. The compositions disclosed herein can also be formulated for intradermal, intralesional, intravitreal, intraocular, and/or subcutaneous administration. In particular embodiments, the compositions can be in the form of, e.g., gels, ointments, pastes, lotions, creams, sprays, foams, or powders.

In other embodiments, the compositions disclosed herein may be formulated for injection, including subcutaneous, subdermal, and/or intraocular. U.S. Pat. No. 7,918,824 discloses syringes suitable for subject use. The compositions for injection can take such forms as suspensions, solutions or emulsions in oily or aqueous vehicles, and can contain formulatory agents such as suspending, stabilizing, preserving and/or dispersing agents. Injectable formulations include one or more compositions disclosed herein in combination with one or more pharmaceutically acceptable sterile isotonic aqueous or non-aqueous solutions, emulsions, or sterile powders which may be reconstituted into sterile injectable solutions or dispersions just prior to use, which may contain antioxidants, buffers, bacteriostats, or solutes.

For injection, compositions can be formulated as aqueous solutions, such as in buffers including Hanks' solution, Ringer's solution, or physiological saline. The aqueous solutions can contain formulatory agents such as suspending, stabilizing, and/or dispersing agents. Examples of suitable aqueous and non-aqueous carriers, which may be employed in the injectable formulations include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of selected particle size in the case of dispersions, and by the use of surfactants.

Injectable formulations may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the pharmaceutical compositions.

Alternatively, the administration form can be in lyophilized and/or provided in powder form for constitution with a suitable vehicle, e.g., sterile pyrogen-free water, before use. Lyophilized compositions can include less than 5% water content; less than 4.0% water content; or less than 3.5% water content.

In another embodiment, the composition can be in a unit dosage form, such as in a suitable diluent in sterile, hermetically sealed ampoules or sterile syringes.

A gel is a substantially dilute cross-linked system, which exhibits no flow when in the steady-state. Most gels are liquid; however, they behave more like solids due to the three-dimensional cross-linked network within the liquid. Gels can have properties ranging from soft and weak to hard and tough.

An ointment is a homogeneous, viscous, semi-solid preparation, most commonly a greasy, thick oil (oil 80%-water 20%) with a high viscosity. Ointments have a water number, which is the maximum quantity of water that 100 g of a base can contain at 20° C.

A paste includes three agents-oil, water, and powder, one of which includes a therapeutic agent. Pastes can be an ointment in which a powder is suspended.

A lotion also includes oil, water, and powder, but can have additional components (e.g., alcohol to hold the emulsion together) and often has a lower viscosity than a paste.

A cream is an emulsion of oil and water in approximately equal proportions. Creams are thicker than lotions and maintain their shape when removed from a container.

Topical formulations disclosed herein can include components, such as animal and vegetable fats, oils, waxes, paraffins, starch, tragacanth, cellulose derivatives, polyethylene glycols, silicones, bentonites, silicic acid, talc and zinc oxide, or mixtures thereof. In various embodiments, topical formulations may include thickening agents, surfactants, organic solvents, tonicity modifiers,

In various embodiments, topical formulations can be prepared using thickening agents, such as carboxymethylcellulose sodium, sodium starch glycollate type C, or Carbomers such as Carbopol® (Lubrizol Advanced Materials, Inc. Cleveland, OH, USA) 934, 980, 981, 1382, 5984, or 2984. In various embodiments, topical formulations can be prepared using surfactants, such as Pluronic® (BASF Corporation, Mount Olive, NJ, USA) co-polymers, such as Pluronic® F-127, and/or a Pluronic® co-polyer having the formula

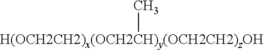

or H [OCH2CH2]49 [OCHCH2]67 [OCH2CH2]49OH; propyl glycol, polypropylene glycol (PPG) stearyl ethers, such as PPG ethers of stearyl alcohol including PPG-20 methyl glucose ether distearate, PPG-15 Stearyl Ether, and PPG-11 Stearyl Ether.

In various embodiments, topical formulations such as gel formulations may include an organic solvent (e.g. a lower alkyl alcohol, such as ethyl alcohol or isopropyl alcohol; a ketone, such as acetone or N-methyl pyrrolidone; a glycol, such as propylene glycol; and the like, or mixtures thereof) present in an amount of 1% to 99%. In particular embodiments, an organic solvent may be present in an amount of 60% to 80%. In various embodiments, topical formulations may include a cellulose derivative, such as hydroxyl ethyl cellulose, hydroxy propyl cellulose, hydroxy propyl methyl cellulose, methyl cellulose, carboxy methyl cellulose, sodium carboxy methyl cellulose, ethyl cellulose, and the like, or combinations thereof present in an amount of 0.1% to 20%. In particular embodiments, a cellulose derivative may be present in an amount of 0.5% to 5%.

In various embodiments, topical formulations such as gel formulations include any suitable tonicity modifier. Exemplary suitable tonicity modifiers include sodium chloride, potassium chloride, mannitol, sucrose, lactose, fructose, maltose, dextrose, dextrose anhydrous, propylene glycol, and glycerol. In various embodiments, the tonicity modifier can be present in an amount of 0.5% to 1% by weight. In particular embodiments, a tonicity modifier can be present in an amount of 0.8% to about 1% by weight of the topical formulation. In various embodiments, buffers can be present in topical formulations. Exemplary buffers include phosphate buffered saline (PBS) acetate buffers, such as sodium acetate trihydrate or glacial acetic acid; and citrate buffers, such as sodium citrate dihydrate and citric acid.

In some embodiments, topical formulations such as gel formulations may have a viscosity of at least 1,000 centipoise (cps). In other embodiments, topical formulations such as gel formulations may have a viscosity of at least about 3,000 cps. In specific embodiments, the viscosity of topical formulations will not exceed 50,000 cps.

Powders and sprays particularly may benefit from the inclusion of excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane. The compositions of the disclosure can be alternatively administered by aerosol. This is accomplished by preparing an aqueous aerosol, liposomal preparation, or solid particles containing a composition of the disclosure. A non-aqueous (e.g., fluorocarbon propellant) suspension also could be used. Sonic nebulizers can be preferred because they minimize exposing the compositions to shear, which can result in degradation of the composition.

Compositions can also be incorporated into wound dressings (e.g., bandages, adhesive bandages, transdermal patches). Generally, in these embodiments, compositions are embedded within puffs, gauzes, fleeces, gels, powders, sponges, or other materials that are associated with a second layer to form a wound dressing. Absorption enhancers can also be used to increase the flux of the composition, and particularly the administration form within the composition, across epithelial cells. The rate of such flux can be controlled by either providing a rate-controlling membrane or dispersing the administration form in a polymer matrix or gel.

In particular embodiments, the second layer of a wound dressing can be an elastomeric layer, vapor-permeable film, waterproof film, a woven or nonwoven fabric, mesh, or the like. The composition containing layer and second layer can be bonded using any suitable method (e.g., the application of adhesives, such as pressure sensitive adhesives, hot melt adhesives, curable adhesives; the application of heat or pressure, such as in lamination; a physical attachment through the use of stitching, studs, other fasteners; or the like).

Wound dressings may include adhesives for attachment to the skin or other tissue. Although any adhesive suitable for forming a bond with the skin or other tissue can be used, in certain embodiments a pressure sensitive adhesive is used. Pressure sensitive adhesives are generally defined as adhesives that adhere to a substrate when a light pressure is applied but leave little to no residue when removed. Pressure sensitive adhesives include solvent in solution adhesives, hot melt adhesives, aqueous emulsion adhesives, calenderable adhesives, and radiation curable adhesives.

The most commonly used elastomers in pressure sensitive adhesives can include natural rubbers, styrene-butadiene latexes, polyisobutylene, butyl rubbers, acrylics, and silicones. In particular embodiments, acrylic polymer or silicone-based pressure sensitive adhesives can be used. Acrylic polymers can often have a low level of allergenicity, be cleanly removable from skin, possess a low odor, and exhibit low rates of mechanical and chemical irritation. Medical grade silicone pressure sensitive adhesives can be chosen for their biocompatibility.

Amongst the factors that influence the suitability of a pressure sensitive adhesive for use in wound dressings of particular embodiments is the absence of skin irritating components, sufficient cohesive strength such that the adhesive can be cleanly removed from the skin, ability to accommodate skin movement without excessive mechanical skin irritation, and good resistance to body fluids.

In particular embodiments, the pressure sensitive adhesive can include a butyl acrylate. While butyl acrylate pressure sensitive adhesives can generally be used for many applications, any pressure sensitive adhesive suitable for bonding skin can be used. Such pressure sensitive adhesives are well known in the art.

In some cases, in order to prolong the effect of a composition, it is desirable to slow the absorption of the composition following injection. Compositions can be formulated as sustained-release systems utilizing semipermeable matrices of solid polymers containing at least one administration form. Various sustained-release materials have been established and are well known by those of ordinary skill in the art. Sustained-release systems may, depending on their chemical nature, release active ingredients following administration for a few weeks up to over 100 days.

In various embodiments, delayed absorption can be accomplished by dissolving or suspending the composition in an oil vehicle. In various embodiments, administration forms can be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts. In addition, prolonged absorption of the injectable pharmaceutical form may be brought about by the inclusion of agents which delay absorption such as aluminum monostearate and gelatin.

Injectable depot forms can be made by forming microencapsule matrices of administration forms in biodegradable polymers such as polylactide-polyglycolide. Depending on the ratio of administration form to polymer, and the nature of the particular polymer employed, the rate of administration form release can be controlled. Examples of other biodegradable polymers include poly(orthoesters) and poly(anhydrides). Injectable depot formulations are also prepared by entrapping the administration form in liposomes or microemulsions which are compatible with body tissue.

Alternatively, delayed absorption of a composition can be accomplished by the use of a liquid suspension of crystalline or amorphous material having poor water solubility. The rate of absorption of the composition then depends upon its rate of dissolution which, in turn, may depend upon crystal size and crystalline form.

Any composition disclosed herein can advantageously include any other pharmaceutically acceptable carriers which include those that do not produce significantly adverse, allergic, or other untoward reactions that outweigh the benefit of administration, whether for research, prophylactic, and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

In some embodiments, the pharmaceutical compositions can include, for example, 25 μg/mL-5 mg/mL, 50 μg/mL-5 mg/mL, 100 μg/mL-5 mg/mL, 150 μg/mL-5 mg/mL, 200 μg/mL-5 mg/mL, 250 μg/mL-5 mg/mL, 300 μg/mL-5 mg/mL, 350 μg/mL-5 mg/mL, 400 μg/mL-5 mg/mL, 450 μg/mL-5 mg/mL, 500 μg/mL-5 mg/mL, 550 μg/mL-5 mg/mL, 600 μg/mL-5 mg/mL, 650 μg/mL-5 mg/mL, 700 μg/mL-5 mg/mL, 750 μg/mL-5 mg/mL, 800 μg/mL-5 mg/mL, 850 μg/mL-5 mg/mL, 900 μg/mL-5 mg/mL, 950 μg/mL-5 mg/mL, 1 mg/mL-5 mg/mL, 1.5 mg/mL-5 mg/mL, 2 mg/mL-5 mg/mL, 2.5 mg/mL-5 mg/mL, 3 mg/mL-5 mg/mL, 3.5 mg/mL-5 mg/mL, 4 mg/mL-5 mg/mL, 4.5 mg/mL-5 mg/mL, 25 μg/mL-2.5 mg/mL, 50 μg/mL-2.5 mg/mL, 100 μg/mL-2.5 mg/mL, 150 μg/mL-2.5 mg/mL, 200 μg/mL-2.5 mg/mL, 250 μg/mL-2.5 mg/mL, 300 μg/mL-2.5 mg/mL, 350 μg/mL-2.5 mg/mL, 400 μg/mL-2.5 mg/mL, 450 μg/mL-2.5 mg/mL, 500 μg/mL-2.5 mg/mL, 550 μg/mL-2.5 mg/mL, 600 μg/mL-2.5 mg/mL, 650 μg/mL-2.5 mg/mL, 700 μg/mL-2.5 mg/mL, 750 μg/mL-2.5 mg/mL, 800 μg/mL-2.5 mg/mL, 850 μg/mL-2.5 mg/mL, 900 μg/mL-2.5 mg/mL, 950 μg/mL-2.5 mg/mL, 1 mg/mL-2.5 mg/mL, 1.5 mg/mL-2.5 mg/mL, 2 mg/ml-2.5 mg/mL, 25 μg/mL-1 mg/mL, 50 μg/mL-1 mg/mL, 100 μg/mL-1 mg/mL, 150 μg/mL-1 mg/mL, 200 μg/mL-1 mg/mL, 250 μg/mL-1 mg/mL, 300 μg/mL-1 mg/mL, 350 μg/mL-1 mg/mL, 400 μg/mL-1 mg/mL, 450 μg/mL-1 mg/mL, 500 μg/mL-1 mg/mL, 550 μg/mL-1 mg/mL, 600 μg/mL-1 mg/mL, 650 μg/mL-1 mg/mL, 700 μg/mL-1 mg/mL, 750 μg/mL-1 mg/mL, 800 μg/mL-1 mg/mL, 850 μg/mL-1 mg/mL, 900 μg/mL-1 mg/mL, 950 μg/mL-1 mg/mL, 25 μg/mL-750 μg/mL, 50 μg/mL-750 μg/mL, 100 μg/mL-750 μg/mL, 150 μg/mL-750 μg/mL, 200 μg/mL-750 μg/mL, 250 μg/mL-750 μg/mL, 300 μg/mL-750 μg/mL, 350 μg/mL-750 μg/mL, 400 μg/mL-750 μg/mL, 450 μg/mL-750 μg/mL, 500 μg/mL-750 μg/mL, 550 μg/mL-750 μg/mL, 600 μg/mL-750 μg/mL L, 650 μg/mL-750 μg/mL, 700 μg/mL-750 μg/mL, 25 μg/mL-500 μg/mL, 50 μg/mL-500 μg/mL, 100 μg/mL-500 μg/mL, 150 μg/mL-500 μg/mL, 200 μg/mL-500 μg/mL, 250 μg/mL-500 μg/mL, 300 μg/mL-500 μg/mL, 350 μg/mL-500 μg/mL, 400 μg/mL-500 μg/mL, 450 μg/mL-500 μg/mL, 25 μg/mL-250 μg/mL, 50 μg/mL-250 μg/mL, 100 μg/mL-250 μg/mL, 150 μg/mL-250 μg/mL, 200 μg/mL-250 μg/mL, 25 μg/mL-100 μg/mL, or 50 μg/mL-100 μg/mL of the administration form.
(iii) Alternative Up-Regulation Methods. Generally, combination treatments disclosed herein will be administered by systemic or local administration of a composition including both Tβ4 and VIP or active fragments or precursors of Tβ4 and/or VIP. However, up-regulating Tβ4 and VIP is not limited to such administrable compositions.

The presence or activity of a protein can be up-regulated by one or more of: increasing the expression of the protein; administering or expressing a more active variant of the protein, reducing degradation of the protein following expression, etc. To cause an up-regulation through increased expression of a protein, the copy number of its gene or genes encoding the protein may be increased. Alternatively, a strong and/or inducible promoter may be used to direct the expression of the gene, the gene being expressed either as a transient expression vehicle, or homologously or heterologously incorporated into a genome. In another embodiment, the promoter, regulatory region, and/or the ribosome binding site upstream of the gene can be altered to achieve over-expression. The expression may also be enhanced by increasing the relative half-life of the messenger or other forms of RNA. Similar mechanisms can be used to up-regulate the expression of genes, for example, genes encoding Tβ4 and VIP.

In particular embodiments, Tβ4 and VIP can be up-regulated through genetic manipulations. For example, nucleotide sequences encoding Tβ4 and VIP can be readily deduced by one of ordinary skill in the art.

In particular embodiments, Tβ4 and/or VIP is up-regulated by administering a vector including a nucleotide sequence that encodes for and directs expression of Tβ4 and/or VIP. The nucleotide sequence can also direct expression of a Tβ4 and/or VIP prodrug or a protein or other molecule that stimulates a cell to produce or activate Tβ4 and/or VIP.

Viral vectors are usually non-replicating or replication-impaired vectors, which means that the viral vector cannot replicate to any significant extent in normal cells (e.g., normal human cells), as measured by conventional means (e.g. via measuring DNA synthesis and/or viral titer). Non-replicating or replication-impaired vectors may have become so naturally (i.e., they have been isolated as such from nature) or artificially (e.g., by breeding in vitro or by genetic manipulation). There will generally be at least one cell-type in which the replication-impaired viral vector can be grown—for example, modified vaccinia Ankara (MVA) can be grown in CEF cells. Typically, viral vectors are incapable of causing a significant infection in a subject, typically in a mammalian subject.

In particular embodiments, the vector is selected from an adenovirus or a poxvirus vector. Examples of viral vectors that are useful in this context include attenuated vaccinia virus vectors such as modified vaccinia Ankara (MVA) and NYVAC, or strains derived therefrom. Other examples of vectors include an avipox vector, such as a fowlpox vectors (e.g., FP9) or canarypox vectors (e.g., ALVAC and strains derived therefrom). Alternative viral vectors include adenoviral vectors (e.g., non-human adenovirus vectors), alphavirus vectors, flavivirus vectors, herpes viral vectors (e.g., herpes simplex, CMV and EBV), influenza virus vectors and retroviral vectors.

In particular embodiments, the vector is a human adenovirus. In another embodiment, the vector is a simian adenovirus. In another embodiment, the vector is a chimpanzee adenovirus. A chimpanzee as referred to herein may include Pan troglodytes (common chimpanzee) and Pan paniscus (Bonobo). In particular embodiments, the vector is selected from adenovirus 5 (Ad5), adenovirus 35 (Ad35), adenovirus 11 (Ad11), adenovirus 26 (Ad26), adenovirus 48 (Ad48) or adenovirus 50 (Ad50).

Additional information about retroviral vectors can be found in, for example, Miller et al., 1993, Meth. Enzymol. 217:581-599; Boesen et al., 1994, Biotherapy 6:291-302, Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114; additional information about adenoviruses can be found in, for example, Kozarsky and Wilson, 1993, Current Opinion in Genetics and Development 3:499-503, Rosenfeld et al., 1991, Science 252:431-434; Rosenfeld et al., 1992, Cell 68:143-155; and Mastrangeli et al., 1993, J. Clin. Invest. 91:225-234; additional information about adena-associated viruses, mammalian artificial chromosomes can be found in, for example, Vos, 1998, Curr. Op. Genet. Dev. 8:351-359; additional information about triplex DNA can be found in, for example, Chan and Glazer, 1997, J. Mol. Med. 75:267-282; and additional information about ribozymes can be found in, for example, Branch and Klotman, 1998, Exp. Nephrol. 6:78-83.

Expression of vectors may be controlled following administration to a subject. In particular embodiments, the desired gene recombinantly expressed in the subject includes an inducible promoter operably linked to the coding region, such that expression of the recombinant gene is controllable by controlling the presence or absence of the appropriate inducer of transcription.

For example, a number of cell- or tissue-specific promoters are known. To accommodate required flexibility in disparate levels and timing of expression such genes are driven from low basal promoters (i.e. TK), or through controlled induction from a Tet on/off promoter. The Tet promoter system benefits from the use of innocuous antibiotic analogs such as anhydrotetracycline, which activates the Tet promoter at concentrations 2 logs lower than with tetracycline, does not result in dysregulation of intestinal flora, does not result in resistance to polyketide antibiotics, and does not exhibit antibiotic activity. Anhydrotetracycline is fully soluble in water, and can be administered in drinking rations to potentiate activation of selected genes in transfected cells. The potential toxicity of anhydrotetracycline, the first breakdown product of tetracycline in the human body, can be circumvented by administration of other analogs, such doxycycline, an FDA-approved tetracycline analog that also activates the Tet on/off promoter system. This system can be employed in the design of a failsafe “kill switch” by tightly regulating inducible expression of a potent pro-apoptotic gene (e.g. Bax) to initiate targeted apoptosis of transfected cells in the event of untoward side effects or when the desired therapeutic endpoint has been achieved. Recent advances in the Tet-on system have resulted in much enhanced repression of promoter leakiness and responsiveness to Dox at concentrations up to 100-fold lower than in the original Tet system (Tet-On Advanced™, Tet-On 3G™). For additional information on TET systems, see, for example, Bujard & Gossen (1992). Proc. Natl. Acad. Sci. U.S.A. 89 (12): 5547-51; Urlinger et al., (2000). Proc. Natl. Acad. Sci. U.S.A. 97 (14): 7963-8; and Zhou et al., (2006). Gene Ther. 13 (19): 1382-1390.

The GAL4-UAS system may also be used. For additional information on GAL4-UAS systems, see, for example, Brand & Perrimon (Jun. 1, 1993). Development 118:401-415; Duffy (2002). Genesis 32:1-15; Janice et al., (1988). Nature (6167): 853-6; Webster et al., (1988). Cell 52 (2): 169-78; Liu & Lehman (2008). Proc Natl Acad Sci UAS 99 (3): 1377-82; Davison et al., (2007). Developmental Biology 304 (2): 811-24; Suster, et al., (2004). Genesis (Wiley Online Library) 39 (4): 240-245; and Luan et al., (2006). Neuron (Elsevier) 52 (3): 425-436.

In particular embodiments, vectors used within the current disclosure do not stimulate vector-derived immunity that would prevent a subsequent use of the disclosed treatments in a subject. This benefit can be confirmed by designing a sensitive assay to detect immune responses (antibody ELISA and T-cell based assays) to components of the treatment.

Particular embodiments utilize adeno-associated virus (AAV). AVV is a small virus which infects humans and some other primate species. AAV is not currently known to cause disease. The virus causes a very mild immune response, lending further support to its apparent lack of pathogenicity. Gene therapy vectors using AAV can infect both dividing and quiescent cells and persist in an extrachromosomal state without integrating into the genome of the host cell. The AVV system also utilizes the Tet-On/Off system as an additional precaution.

Administration methods can also include nanoparticle gene transfer technology to deliver Tet On-controlled Tβ4 and/or VIP cDNA into wounded skin cells.

Administration methods can also include impalefection technology to deliver Tβ4 and/or VIP expression DNA vector to wounded skin cells. Impalefection is a method of gene delivery using nanomaterials, such as carbon nanofibers, carbon nanotubes, and nanowires. One of the features of impalefection is spatially resolved gene delivery that holds potential for tissue engineering approaches in wound healing as gene activated matrix technology (J Regener Med 2000, 1:25-29).

Administration methods can also include cell therapy or engineered skin layer technology. An AAV-Tb4 and/or VIP can be transfected into epithelial cells or ex vivo cultured corneal layer that can be applied. By utilizing the teachings of the disclosure, epithelial cells or engineered corneal tissues with controlled expression of Tβ4 and/or VIP represent a very promising therapeutic approach for treating ocular conditions.

Purified human Tβ4 and/or VIP protein also can be delivered into ocular cells through gold nanoparticle-mediated laser transfection (Laser-Gold Nanoparticle Technology: Nanotechnology 2014, 25:245101).

Genetic therapies can be achieved using any method known in the art, including transfection, electroporation, microinjection, lipofection, calcium phosphate mediated transfection, infection with a viral or bacteriophage vector containing the gene sequences, cell fusion, chromosome-mediated gene transfer, microcell-mediated gene transfer, sheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present disclosure.

Targeted genetic engineering systems can also be used. The CRISPR (Clustered

Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9 (8): 805-7; Ramirez et al., Nucl Acids Res, 2012, 40 (12): 5560-8; Kim et al., Genome Res, 2012, 22 (7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. For information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14 (I): 49-55; Beurdeley et al., Nat Commun, 2013, 4:1762; Scharenberg et al., Curr Gene Ther, 2013, 13 (4): 291-303; Gaj et al., Nat Methods, 2012, 9 (8): 805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a constructs into cells.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7, 148,203; 8,227,432; and 9,228, 180.

As is understood by one of ordinary skill in the art, “up-regulation” can be measured against a relevant control condition. For example, an up-regulation of Tβ4 and VIP can be measured by comparing an Tβ4 and VIP level to that observed in a wound area of a diabetic subject that has not received a treatment disclosed herein.

(iv) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with administration forms disclosed herein including salts and prodrugs thereof. Treating subjects includes delivering therapeutically effective amounts. Therapeutically effective amounts include those that provide effective amounts, prophylactic treatments, and/or therapeutic treatments.

An “effective amount” is the amount of a combination treatment necessary to result in a desired physiological change in the subject. Effective amounts are often administered for research purposes. Effective amounts disclosed herein can promote epithelial integrity and/or treat ocular disorders.

A “prophylactic treatment” includes a treatment administered to a subject who displays signs or symptoms of a condition or displays only early signs or warning symptoms for the condition such that treatment is administered for the purpose of diminishing, preventing, or decreasing the risk of the condition developing further. Thus, a prophylactic treatment functions as a preventative treatment against a condition. A prophylactic treatment also can be administered to subjects at risk for developing a condition. For example, in subjects at risk for developing chronic wounds, prophylactic treatments can be administered at the time a wound occurs or as soon as is reasonably or practically possible thereafter. Diabetic subjects are one group of subjects at risk for developing chronic wounds. Other subjects at risk for developing chronic wounds include those who suffer from an inflammatory condition.

A “therapeutic treatment” includes a treatment administered to a subject who has a condition and is administered to the subject for the purpose of promoting the healing of a condition. Therapeutic treatments can promote epithelial integrity and/or treat ocular disorders.

Function as an effective amount, a prophylactic treatment, or a therapeutic treatment are not mutually exclusive and an administration to a subject can achieve more than one.

In particular embodiments, a treated condition is a wound. A “wound” refers to open wounds, such as incisions, lacerations, abrasions, avulsions, puncture wounds, penetration wounds, gunshot wounds, burn wounds, thermal burns, chemical burns, electrical burns, and radiation burns. Wounds also include pressure wounds. As used herein, “wound” also includes internal injuries, such as retinal or macular tears. “Chronic wounds” include wounds that take longer to heal than would be expected by a physician. In diabetics, “chronic wounds” include wounds that take longer to heal as compared to a wound of a healthy control subject. For example, a corneal incision wound is expected to heal within 42 hours following its occurrence in a healthy control subject.

The present disclosure describes compositions and methods to promote wound healing. In particular embodiments, the compositions and methods can be used to reduce the occurrence of chronic wounds. In particular embodiments, the compositions and methods can be used to promote wound healing in diabetic subjects. In particular embodiments, the compositions and methods can be used to reduce the occurrence of chronic wounds in diabetic subjects.

Wound healing generally can be divided into three steps: re-epithelialization, granulation, and neovascularization. Delayed re-epithelialization and inadequate formation of granulation tissue can lead to the development of chronic wounds. Endothelial progenitor cells (EPCs), which derive from bone marrow, normally travel to sites of injury and are essential for the formation of blood vessels and wound healing.

Objective measures for the promotion of wound healing include the time required for the closure of an open wound or establishment of a biological barrier, or according to the methods described in Experimental Example 1. For example, diabetic subjects provided with a combination treatment disclosed herein will demonstrate faster wound healing than diabetic subjects with a similar wound who do not receive a treatment disclosed herein.

Particular embodiments of the present disclosure promote eye health. Diabetic retinopathy is caused by chronically high blood sugar from diabetes and is associated with damage to the tiny blood vessels in the retina. Diabetic retinopathy can cause blood vessels in the retina to leak fluid or hemorrhage, distorting vision. In its most advanced stage, new abnormal blood vessels proliferate on the surface of the retina, which can lead to scarring and cell loss in the retina. Diabetic retinopathy can progress through four stages: 1) Mild nonproliferative retinopathy in which small areas of balloon-like swelling in the retina's tiny blood vessels, called microaneurysms, occur at this earliest stage of the disease. These microaneurysms may leak fluid into the retina. 2) Moderate nonproliferative retinopathy which, as the disease progresses, blood vessels that nourish the retina may swell and distort. They may also lose their ability to transport blood. Both conditions cause characteristic changes to the appearance of the retina and may contribute to diabetic macula edema. 3) Severe nonproliferative retinopathy in which many more blood vessels are blocked, depriving blood supply to areas of the retina. These areas secrete growth factors that signal the retina to grow new blood vessels. 4) Proliferative diabetic retinopathy (PDR): At this advanced stage, growth factors secreted by the retina trigger the proliferation of new blood vessels, which grow along the inside surface of the retina and into the vitreous gel. The new blood vessels are fragile, which makes them more likely to leak and bleed. Accompanying scar tissue can contract and cause retinal detachment. Retinal detachment can lead to permanent vision loss.

Diabetic macular edema is the build-up of fluid in the macula in diabetics and is associated with diabetic retinopathy. Diabetic retinopathy damages the blood vessels in the retina. Left untreated, these blood vessels begin to build up pressure in the eye and leak fluid, causing diabetic macular edema. Common symptoms of diabetic macular edema are blurry vision, floaters, double vision, and eventually blindness if left untreated.

Corneal ulcer, or ulcerative keratitis, is an inflammatory or more seriously, infective condition of the cornea involving disruption of its epithelial layer with involvement of the corneal stroma. A corneal ulcer is essentially an open sore. Corneal ulcers are extremely painful due to nerve exposure, and can cause tearing, squinting, and vision loss of the eye. There may also be signs of anterior uveitis, such as miosis (small pupil), aqueous flare (protein in the aqueous humour), and redness of the eye. Corneal ulcers can be caused by trauma or by infective microorganisms such as bacteria, fungi, protozoans and viruses.

Optic neuritis is inflammation of the optic nerve, which is the nerve that carries visual signals from the eye to the brain. The condition may cause pain and sudden, reduced vision, and/or blurry vision in the affected eye(s). Other early symptoms can be reduced night vision, sensitivity to light (photophobia) and red eyes. Some common causes of optic neuritis are multiple sclerosis and blood clots. Optic neuritis may also be caused by autoimmune disease and diabetes.

Macular edema is the build-up of fluid in the macula. Fluid buildup causes the macula to swell and thicken, which distorts vision. Macular edema is typically caused by increased leakage from damaged retinal blood vessels or growth of abnormal blood vessels in the deep retina. Macular edema may also be caused by inflammatory processes. Macular edema may commonly be associated with diabetes. Age-related macular degeneration may also cause macular edema.

Optic nerve degeneration aka optic atrophy refers to a group of conditions in which the optic nerve is damaged: some are genetically-based while others are due to trauma, toxins, deficiencies, and inflammation. In optic nerve degeneration the optic nerve is limited in its capacity to transmit accurate information about visual input in the form of electrical impulses to the brain. Symptoms can include blurred vision, decrease in visual acuity, decreases in peripheral vision, decrease in color vision, decrease in contrast sensitivity, and poor constriction of the pupil when exposed to light. Some causes of optic nerve degeneration are glaucoma, diabetes, stroke of the optic nerve aka ischemic optic neuropathy, a tumor pressing on the optic nerve, and optic neuritis.

Dry eye occurs when the quantity and/or quality of tears fails to keep the surface of the eye adequately lubricated. The risk of developing dry eye increases with advancing age. Dry eye causes a scratchy sensation or the feeling that something is in the eye. Other symptoms include stinging or burning, episodes of excess tearing that follow periods of dryness, discharge, pain, and redness in the eye. Dry eye can occur when basal tear production decreases, tear evaporation increases, or tear composition is imbalanced. Dry eye can be caused by: medications including antihistamines, decongestants, antidepressants, birth control pills, hormone replacement therapy to relieve symptoms of menopause, and medications for anxiety, Parkinson's disease, and high blood pressure; rosacea (an inflammatory skin disease) and blepharitis (an inflammatory eyelid disease); autoimmune disorders such as Sjogren's syndrome, lupus, scleroderma, and rheumatoid arthritis and other disorders such as diabetes, thyroid disorders, and Vitamin A deficiency; Seasonal allergies; and windy, smoky, or dry environments can increase tear evaporation.

Age Related Macular Degeneration (AMD or ARMD) is a common eye condition and a leading cause of vision loss among people age 50 and older. It causes damage to the macula, a small spot near the center of the retina and the part of the eye needed for sharp, central vision. The presence of medium-to-large drusen, which are white or yellow deposits beneath the retina, may indicate AMD. There are three stages of AMD defined in part by the size and number of drusen under the retina: 1) Early AMD which is diagnosed by the presence of medium-sized drusen, which are about the width of an average human hair.

People with early AMD typically do not have vision loss. 2) People with intermediate AMD typically have large drusen, pigment changes in the retina, or both. These changes can only be detected during an eye exam. Intermediate AMD may cause some vision loss, but most people will not experience any symptoms. 3) Late AMD. In addition to drusen, people with late AMD have vision loss from damage to the macula. There are two types of late AMD: A) Dry AMD (also called geographic atrophy, atrophic AMD, non-neovascular AMD, or non exudative AMD), wherein there is a gradual breakdown or thinning of the light-sensitive cells in the macula that convey visual information to the brain and of the supporting tissue beneath the macula (photoreceptors, retinal pigment epithelium, choriocappillaris). These changes cause vision loss. B) Neovascular AMD (also called wet AMD), wherein abnormal blood vessels grow in the choroid layer underneath the retina. These vessels can leak fluid and blood, which may lead to swelling and damage of the macula. The damage may be rapid and severe, unlike the more gradual course of geographic atrophy. It is possible to have both geographic atrophy and neovascular AMD in the same eye, and either condition can appear first.

Juvenile macular degeneration is a series of inherited eye disorders that affects children and young adults. Juvenile macular degeneration is different from age-related macular degeneration, which occurs as part of the body's natural aging process. Juvenile macular degeneration is sometimes called juvenile macular dystrophy. They include Stargardt's disease, Best disease, and juvenile retinoschisis. They can cause central vision loss that often starts in childhood or young adulthood.

Stargardt disease is an inherited disorder of the retina. Stargardt disease is also called Stargardt macular dystrophy, or fundus flavimaculatus. The disease causes progressive damage—or degeneration—of the macula, The disease typically causes vision loss during childhood or adolescence, although in some forms, vision loss may not be noticed until later in adulthood. Vision loss is due to abnormal accumulation of a fatty yellow pigment (lipofuscin) in the cells within the macula. People with Stargardt disease also have problems with night vision, and some have problems with color vision. The signs and symptoms of Stargardt disease typically appear in late childhood to early adulthood and worsen over time. The most common cause is believed to be by mutations in the ABCA4 gene.

Best disease (vitelliform macular dystrophy) (BVMD) is a slowly progressive form of macular degeneration. It usually begins in childhood or adolescence, but age of onset and severity of vision loss can vary. Affected people first have normal vision, followed by decreased central visual acuity and distorted vision (metamorphopsia). Peripheral vision is not affected. BVMD is characterized by atrophy of the retinal pigment epithelium and impaired central visual function.

Adult-onset vitelliform macular dystrophy (AVMD) is an eye disorder that can cause progressive vision loss. AVMD affects an area of the retina called the macula, which is responsible for sharp central vision. The condition causes fatty yellow pigment to accumulate in cells underlying the macula, eventually damaging the cells.

Juvenile retinoschisis is an eye condition characterized by impaired vision that begins in childhood and occurs almost exclusively in males. The condition affects the retina and affects the sharpness of vision. Central vision is more commonly affected. Vision often deteriorates early in life, but then usually becomes stable until late adulthood. A second decline in vision typically occurs in a person's fifties or sixties.

Light induced retinal damage (LIRD) resembles many features of several retinal degenerative diseases, particularly age-related macular degeneration. Photochemical damage is the most common form of LIRD and occurs when light is absorbed by a chromophore and leads to the formation of an electronically excited state of that molecule, which then undergoes either chemical transformation itself and/or interacts with other molecules leading to chemical changes of both interacting molecules. Light damage in the human retina due to excessive exposure to sunlight is known as solar retinopathy. Photochemical damage typically damages the rods and cones of the eye.

Doyne honeycomb retinal dystrophy is a condition that affects the eyes and causes vision loss. It is characterized by small, round, white drusen that accumulate beneath the retinal pigment epithelium (the pigmented layer of the retina). Over time, drusen may grow and come together, creating a honeycomb pattern. It usually begins in early to mid-adulthood, but the age of onset can vary. The degree of vision loss also varies.

Uveitis is the inflammation of the uvea, the pigmented layer that lies between the inner retina and the outer fibrous layer composed of the sclera and cornea. The uvea consists of the iris, the ciliary body and the choroid. The type of uveitis depends on which structure is affected. Iritis (anterior uveitis) affects the front of the eye and is the most common type. Cyclitis (intermediate uveitis) affects the ciliary body. Choroiditis and retinitis (posterior uveitis) affect the back of the eye. Diffuse uveitis (panuveitis) occurs when all layers of the uvea are inflamed. Warning signs often come on suddenly and can get worse quickly. They include eye redness, pain, light sensitivity, floaters and blurred vision. Possible causes of uveitis are infection, injury, or an autoimmune or inflammatory disease.

Scleritis, or inflammation of the sclera, can present as a painful red eye with or without vision loss. Scleritis may be associated with autoimmune disorders, connective tissue disorders and generalized vasculitic abnormalities. Scleritis can also result from an infectious process caused by bacteria including pseudomonas, fungi, mycobacterium, viruses, or parasites. The most common form, anterior scleritis, is defined as scleral inflammation anterior to the extraocular recti muscles. Posterior scleritis is defined as involvement of the sclera posterior to the insertion of the rectus muscles. Anterior scleritis can be subdivided into diffuse, nodular, or necrotizing forms. In the diffuse form, anterior scleral edema is present along with dilation of the deep episcleral vessels. The entire anterior sclera or just a portion may be involved. In nodular disease, a distinct nodule of scleral edema is present. The nodules may be single or multiple in appearance. Necrotizing anterior scleritis is the most severe form of scleritis. Scleritis is characterized by severe pain and extreme scleral tenderness.

Sarcoidosis is a systemic autoinflammatory disease that can affect multiple parts of the body and cause varying levels of inflammation. Ocular sarcoidosis can involve any part of the eye and its adnexal tissues, and may cause uveitis, episcleritis/scleritis, eyelid abnormalities, conjunctival granuloma, optic neuropathy, lacrimal gland enlargement and orbital inflammation. Ocular sarcoidosis can be a “granulomatous” uveitis, i.e., it creates large clumps or collections of inflammatory cells visible on the back of the cornea on exam. Glaucoma and cataracts can be complications from inflammation itself or adverse effects from therapy. Ocular sarcoidosis can manifest itself with blurred vision, photophobia, floaters, redness, and pain from uveitis.

Cone-rod dystrophy is a group of inherited eye disorders that affect the light sensitive cells of the retina, i.e., the cones and rods. People with this condition experience vision loss over time as the cones and rods deteriorate. Initial signs and symptoms that usually occur in childhood may include decreased sharpness of vision (visual acuity) and abnormal sensitivity to light (photophobia). These signs are usually followed by blind spots in the central field of vision (scotomas), loss of color perception, night blindness and loss of peripheral vision. Cone-rod dystrophy can be either autosomal dominant, autosomal recessive or X-linked and may be caused by defects in at least 17 different genes.

Choroidal dystrophies are a group of inherited disorders that involve the choroid and can involve the retina. They include choroideremia, gyrate atrophy, central areolar dystophy, diffuse choroidal atrophy, helicoid peripapillary chorioretinal degeneration and pigmented paravenous retinochoroidal atrophy. Choroideremia is a genetic condition that causes vision loss and typically affects males. The first symptom is usually impairment of night vision (night blindness), which can occur in childhood. People with this disorder also experience narrowing of the field of vision (tunnel vision) and decrease in the ability to see details (visual acuity). The vision problems are due to loss of cells in the retina and choroid. Gyrate atrophy, also known as ornithine aminotransferase deficiency, is an autosomal recessive dystrophy caused by mutations in the gene for ornithine aminotransferase. Symptoms include myopia, often appearing in early childhood, leading to night blindness, limited visual field, and posterior subcapsular cataracts. Symptoms of gyrate atrophy are progressive and can lead to complete blindness by age 45 to 65. Central areolar choroidal dystrophy is a hereditary macular disorder, usually presenting between the ages of 30-60, characterized by a large area of atrophy in the center of the macula and the loss or absence of photoreceptors, retinal pigment epithelium and choriocapillaris in this area, resulting in a progressive decrease in visual acuity. Diffuse choroidal dystrophy is an inherited autosomal dominant disorder that affects the choroid and retina. It is similar to central areolar choroidal dystrophy but is characterized by earlier manifestations of the disease by about ten tears. Symptoms include night vision difficulties and diminishing central and peripheral vision. Helicoid peripapillary chorioretinal degeneration is a autosomal dominantly inherited chorioretinal degeneration disease, presenting at birth or infancy, characterized by progressive bilateral retinal and choroidal atrophy, appearing as lesions on the optic nerve and peripheral ocular fundus and leading to blind spots and central vision loss. Congenital anterior polar cataracts are sometimes associated with this disease. Pigmented paravenous retinochoroidal atrophy is characterized by perivenous aggregations of pigment clumps associated with peripapillary and radial zones of retinochoroidal atrophy that are distributed along the retinal veins. Patients with this disorder may be asymptomatic or may have blurred vision.

Retinitis pigmentosa or retinitis is inflammation of the retina of the eye. Retinitis pigmentosa encompasses a group of genetic disorders that involve a breakdown and loss of cells in the retina. As these cells breakdown and die, patients experience progressive vision loss. The most common feature of all forms of retinitis pigmentosa is a gradual breakdown of rods and cones. Most forms of RP first cause the breakdown of rod cells. These forms of retinitis pigmentosa, sometimes called rod-cone dystrophy, usually begin with night blindness. CMV (cytomegalovirus) retinitis develops from a viral infection of the retina.

Common symptoms of retinitis pigmentosa include difficulty seeing at night and a loss of side (peripheral) vision through progressive degeneration of the retina. As retinitis pigmentosa progresses, the field of vision narrows, a condition known as “tunnel vision,” until only central vision (the ability to see straight ahead) remains.

Usher syndrome is a genetic disorder, inherited as an autosomal recessive trait, characterized by sensorineural hearing loss or deafness and progressive vision loss due to retinitis pigmentosa.

Retinoblastoma is a type of cancer from genetic mutations that forms in the retina (the light-sensitive tissue at the back of the eye). There are two forms, namely, heritable and non-heritable. It usually develops before the age of 5. The most common first sign of retinoblastoma is a visible whiteness in the pupil called “cat's eye reflex” or leukocoria. Other signs and symptoms of retinoblastoma may include crossed eyes or eyes that do not point in the same direction (strabismus), which can cause squinting; a change in the color of the colored part of the eye (iris); redness, soreness, or swelling of the eyelids; and blindness or poor vision in the affected eye or eyes.

Reticular pseudodrusen are small, gray deposits located above the retinal pigment epithelium. In recent years, reticular pseudodrusen have been recognized as a risk factor for the development of late-stage age-related macular degeneration.

Eye floaters are specks or spots that become evident in the field of vision. They can also look like cobwebs. Floaters can be caused by age-related changes to the vitreous humor, i.e., typically shrinkage which causes microscopic collagenous fibers within the vitreous to clump and cast tiny shadows on the retina. Floaters can also be associated with retinal and posterior vitreous detachments.

Eye flashes can occur when the vitreous humor shrinks and pulls on the retina. They can appear as pin pricks or spots of light, shooting stars or can appear as jagged or wavy streaks of light. Other conditions associated with eye flashes are migraines and detached or tom retinas.

Keratoconus is an eye disorder that affects the structure of the cornea. In keratoconus, the shape of the cornea slowly changes shape from round to a cone shape. It also gets thinner and the eye bulges out. In most people, these changes are progressive. In its earliest stages, keratoconus causes slight blurring and distortion of vision and increased sensitivity to glare and light. These symptoms usually appear in the late teens or late 20s. Keratoconus may progress for 10-20 years and then slow in its progression. As keratoconus progresses, the cornea bulges more and vision can become more distorted. In some cases, the cornea will swell and cause a sudden and significant decrease in vision. The swelling occurs when the strain of the cornea's protruding cone-like shape causes one or more small cracks to develop. The swelling may last for weeks or months as the crack heals and is gradually replaced by scar tissue.

Ocular hypertension occurs when the pressure inside the eye (intraocular pressure) is higher than normal. This may be defined as a pressure greater than 21 mm Hg in one or both eyes. In ocular hypertension, the eye does not drain fluid properly. This causes eye pressure to build up. Higher than normal eye pressure can cause glaucoma. However, ocular hypertension is differentiated from glaucoma. With ocular hypertension, the optic nerve looks normal and there are no signs of vision loss. If high pressure causes damage to the optic nerve, it may lead to glaucoma.

Glaucoma is a group of diseases that damage the eye's optic nerve and can result in vision loss and blindness. Eye pressure is a major risk factor for optic nerve damage. Open-angle glaucoma, is the most common form of the disease. Fluid flows continuously in and out of a chamber in front of the eye called the anterior chamber and nourishes nearby tissues. The fluid leaves the chamber at the open angle where the cornea and iris meet. When the fluid reaches the angle, it flows through a spongy meshwork and leaves the eye. In open-angle glaucoma, even though the drainage angle is “open”, the fluid passes too slowly through the meshwork drain. Since the fluid builds up, the pressure inside the eye rises to a level that may damage the optic nerve. When the optic nerve is damaged from increased pressure, open-angle glaucoma—and vision loss may result.

Presbyopia is a common type of vision disorder that occurs with age. It results in the inability to focus up close, a problem associated with refraction in the eye. In presbyopia, the eye is not able to focus light directly on to the retina due to the hardening of the natural lens. Aging also affects muscle fibers around the lens making it harder for the eye to focus on up close objects. The ineffective lens causes light to focus behind the retina, causing poor vision for objects that are up close.

Bietti's Crystalling Dystrophy (BCD) is an inherited eye disease. Symptoms of BCD include: crystals in the cornea; yellow, shiny deposits on the retina; and progressive atrophy of the retina, choriocapillaries and choroid. This tends to lead to progressive night blindness and visual field constriction.

Behqet's disease is an autoimmune disease that causes inflammation in blood vessels. It causes swelling in some parts of the eye. Inflammatory eye disease can develop early in the disease course and lead to permanent vision loss. Ocular involvement can be in the form of posterior uveitis, anterior uveitis, or retinal vasculitis. Anterior uveitis presents with painful eyes, conjuctival redness, hypopyon, and decreased visual acuity, while posterior uveitis presents with painless decreased visual acuity and visual field floaters. A rare form of ocular involvement in this syndrome is retinal vasculitis which presents with painless decrease of vision with the possibility of floaters or visual field defects.

Achromatopsia 2 is a condition that affects the color vision. Most people have complete achromatopsia which is characterized by a total absence of color vision (only able to see black, white and shades of gray). Rarely, affected people may have incomplete achromatopsia which is associated with some color discrimination. Other common signs and symptoms include reduced visual acuity, involuntary back-and-forth eye movements, increased sensitivity to light (photophobia), and hyperopia (farsightedness). Achromatopsia 2 is believed to be caused by changes (mutations) in the CNGA3 gene and is inherited in an autosomal recessive manner.

Acute posterior multifocal placoid pigment epitheliopathy (APMPPE) is an acquired, inflammatory eye condition affecting the retina, retinal pigment epithelium (pigmented layer of the retina), and choroid. It usually affects both eyes and is characterized by multiple, yellow-white lesions in the back of the eye.

Acute zonal occult outer retinopathy is a rare condition that affects the eyes. People with this condition may experience a sudden onset of photopsia (the presence of perceived flashes of light) and an area of partial vision loss (a blindspot). Other symptoms may include “whitening of vision” or blurred vision.

Ocular albinism with late-onset sensorineural deafness (OASD), is a rare, X-linked inherited type of ocular albinism, characterized by severe visual impairment, translucent pale-blue iridies, a reduction in the retinal pigment and moderately severe deafness by middle age.

Alstrom syndrome is a rare genetic disorder that affects many body systems. Symptoms develop gradually, beginning in infancy, and can be variable. In childhood, the disorder is generally characterized by vision and hearing abnormalities, childhood obesity, and heart disease (cardiomyopathy). Vision abnormalities, include cone-rod dystrophy and cataracts.

Amyloid corneal dystrophy, aka gelatinous drop-like corneal dystrophy is a form of superficial corneal dystrophy characterized by multiple prominent milky-white gelatinous nodules beneath the corneal epithelium, photophobia and marked visual impairment.

Anterior ischemic optic neuropathy is an eye disorder characterized by infarction of the optic disk leading to vision loss. It can be nonarteritic (nonarteritic anterior ischemic optic neuropathy) or arteritic, the latter being associated with giant cell arteritis (GCA; often termed temporal arteritis).

Axenfeld-Rieger syndrome is a group of disorders that mainly affects the development of the eye. Common eye symptoms include cornea defects and iris defects. People with this syndrome may have an off-center pupil (corectopia) or extra holes in the eyes that can look like multiple pupils (polycoria). People with this disorder typically have cornea defects. They may have a cloudy cornea or posterior embryotoxin, an opaque ring around the outer edge of the cornea. People with this disorder can also have issues with their iris such as iris stands, which is connective tissue that connects the iris with the lens.

Bardet-Biedl syndrome is an inherited condition that affects many parts of the body. People with this syndrome have progressive visual impairment due to cone-rod dystrophy. Progressive vision loss due to deterioration of the retina. This usually begins in mid-childhood with problems with night vision, followed by the development of blind spots in peripheral vision. Blind spots become bigger with time and eventually merge to produce tunnel vision. Most individuals also develop blurred central vision and become legally blind by adolescence or early adulthood (over 90% of cases).

Behr syndrome is a disorder characterized by early-onset optic atrophy along with neurological features, including ataxia, spasticity, and intellectual disability. People with Behr syndrome typically have visual disturbances (e.g. optic atrophy, nystagmus, scotoma, and bilateral retrobulbar neuritis).

Bietti crystalline comeoretinal dystrophy is an inherited eye disease. Symptoms include crystals in the cornea (the clear covering of the eye); yellow, shiny deposits on the retina; and progressive atrophy of the retina, choriocapillaries and choroid (the back layers of the eye). This tends to lead to progressive night blindness and loss of visual acuity.

Birdshot chorioretinopathy is an eye condition in which painless, light-colored spots develop on the retina. These spots are scattered in a “birdshot” pattern. The effects of this condition on vision are quite variable; some individuals' vision is only mildly affected, whereas others experience a significant decline in vision, the appearance of floaters, night blindness, and other vision problems. Symptoms typically begin around middle age.

Blue cone monochromatism is an inherited vision disorder. In this condition, the light sensitive cells in the eye used for color vision (cones) are affected. There are three types of cones that respond to one of three colors: red, green, and blue. When people have blue cone monochromatism, both the red and green cones do not function properly, while the blue cones work normally.

Coats disease is an eye disorder characterized by abnormal development of the blood vessels in the retina (retinal telangiectasia). Most people begin displaying symptoms in childhood. Early signs and symptoms vary but may include vision loss, “crossed eyes” (strabismus), and a white mass in the pupil behind the lens of the eye (leukocoria). Over time, Coats disease may also lead to retinal detachment, glaucoma, and clouding of the lens of the eye (cataracts).

Iridocorneal endothelial syndrome describes a group of eye diseases that are characterized by three main features: 1) visible changes in the iris (the colored part of the eye that regulates the amount of light entering the eye), 2) swelling of the cornea, and 3) development of glaucoma.

Corneal dystrophy, Avellino type is an inherited condition that affects the stromal or central layer of the cornea. It results in the development of small particles or granules (like breadcrumbs) on the cornea (known as granular corneal dystrophy) and the development of lesions that resemble cracked glass (known as lattice corneal dystrophy). These eye lesions usually develop on the stromal layer before age 20. As affected individuals age, the lesions may become larger, more prominent, and involve the entire stromal layer.

Schnyder corneal dystrophy is a rare form of stromal corneal dystrophy characterized by corneal clouding or crystals within the corneal stroma, and a progressive decrease in visual acuity.

Thiel-Behnke corneal dystrophy is a rare form of superficial corneal dystrophy characterized by sub-epithelial honeycomb-shaped corneal opacities in the superficial cornea, and progressive visual impairment.

Eales disease is a rare vision disorder that appears as an inflammation and white haze around the outercoat of the veins in the retina. This condition is most common among young males and normally affects both eyes. In most cases, vision becomes suddenly blurred because the vitreous seeps out.

Epithelial basement membrane corneal dystrophy is a condition where the epithelium of the cornea (the outermost region of the cornea) loses its normal clarity due to a buildup of cloudy material. This dystrophy occurs when the epithelium's basement membrane develops abnormally, causing the epithelial cells to not properly adhere to it.

Fish-eye disease is a rare ocular condition. People with this condition generally develop corneal clouding beginning in adolescence or early adulthood. Overtime, the condition gradually worsens and can lead to significant vision loss.

Fuchs endothelial corneal dystrophy is an eye disorder that affects the thin layer of cells that line the back part of the cornea (endothelium). It is manifest when these cells slowly start to die off. These cells help pump excess fluid out of the cornea. As more and more cells are lost, fluid begins to build up in the cornea, causing swelling and a cloudy cornea.

Goldmann-Favre syndrome, also known as the severe form of enhanced S-cone syndrome, is an inherited eye disease that affects the retina. Within the retina are “red,” “blue,” and “green” cones for visualizing color; and rods which allows sight in dim light. People with Goldmann-Favre syndrome are born with an overabundance of blue cones, a reduced number of red and green cones, and few, if any, functional rods.

Late-onset retinal degeneration is an inherited retinal dystrophy characterized by delayed dark adaptation and nyctalopia and drusen deposits presenting in adulthood, followed by cone and rod degeneration that presents in the sixth decade of life, which leads to central vision loss.

Leber congenital amaurosis is an eye disorder that primarily affects the retina. People with this condition typically have severe visual impairment beginning in infancy. Other features include photophobia, involuntary movements of the eyes (nystagmus), and extreme farsightedness. The pupils also do not react normally to light. Additionally, the cornea may be cone-shaped and abnormally thin (keratoconus).

Peters anomaly is a disorder of the eye which involves thinning and clouding of the cornea and attachment of the iris to the cornea, which causes blurred vision. It may also be associated with clouding of the lens of the eye (cataracts) or other lens abnormalities.

Punctate inner choroidopathy is an inflammatory disorder that primarily affects the choroid of the eye and occurs predominantly in young, nearsighted (myopic) women. Signs and symptoms may include scotomata, blurred vision, photopsias, floaters, photophobia, distorted vision (metamorphopsia), and/or loss of peripheral vision.

Senior Loken syndrome (SLS) is a rare syndrome that mainly affects the kidneys and eyes. SLS affects the eyes by causing varying degrees of retinal dystrophy, which is inherited progressive wasting of the retina. Some children with SLS have a severe type of retinal dystrophy at birth called Leber congenital amaurosis (LCA). Symptoms of LCA include severe farsightedness, light sensitivity (photophobia), and nystagmus.

Snowflake vitreoretinal degeneration is characterized by the presence of small granular-like deposits resembling snowflakes in the retina, fibrillary vitreous degeneration and cataract.

Visual snow syndrome causes a person to see numerous snow-like flickering tiny dots that fill the entire visual field in both eyes. For most people with the syndrome, the visual snow is always present and occurs in both eyes. The visual snow may worsen at times when the brain and eyes are “tired”, such as after looking at a computer screen for a long time or during times of stress. Other visual symptoms that can be associated with visual snow syndrome include sensitivity to light (photophobia), continuing to see an image after it is no longer in the field of vision (palinopsia), impaired night vision (nyctalopia), and seeing images from within the eye itself (entoptic phenomena), such as seeing small floating objects or flashes of light.

Wagner syndrome is a hereditary eye disorder that leads to progressive vision loss. It is characterized by changes to the vitreous, which it becomes thin and watery and appears empty. The first signs and symptoms usually appear in childhood, but onset may be as early as age 2. Signs and symptoms may include: thinning of the light-sensitive tissue that lines the back of the eye (retinal detachment), abnormalities of the blood vessels within the retina (the choroid), and degeneration of the retina and choroid.

Symptoms of the above-listed eye disorders can include vision loss, drusen, pigment changes in the retina, abnormal blood vessel growth, leaky blood vessels, macular swelling, corneal swelling, corneal thinning, accumulation of a fatty yellow pigment (lipofuscin), night blindness, distorted vision, blurry vision, rod damage, cone damage, uvea inflammation, eye redness, pain, sensitivity to light (photophobia), floaters, eye flashes, nodules, orbital inflammation, lacrimal gland enlargement, decreased visual acuity, decrease in contrast sensitivity, blind spots, loss of color perception, loss of peripheral vision, fluid build-up in the macula, retinal scarring, double vision, pigment clumps, tunnel vision, thin cornea, spotting, leukocoria, lesions, crystals, nystagmus, and any other symptoms associated with the above-listed disorders. It should be understood that each of the above-listed eye disorders may have one or more of the above-listed symptoms.

Eye disorders suitable for treatment herein include diabetic retinopathy, diabetic macular edema, corneal ulcer, Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Beliefs disease, Achromatopsia 2, acute posterior multifocal placoid pigment epitheliopathy (APMPPE), acute zonal occult outer retinopathy (AZOOR), adult-onset vitelliform macular dystrophy (AVMD), ocular albinism with late-onset sensorineural deafness (OASD), Alstrom syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline comeoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome.

Objective measures for the promotion of eye health include epithelial integrity, ocular surface regularity and normal tear secretion. Slit lamp examination of fluorescence staining for epithelial integrity, ocular surface regularity and Schirmer's test uses paper strips inserted into the eye for several minutes to measure the production of tears. Objective measures for the maintenance of vision in humans include no or reduced changes in visual scores (20/20; 20/15; 20/10) as determined by an ophthalmologist.

Corneal sensitivity to touch can be assessed by an aesthesiometer that measures the corneal touch threshold (CTT), which is the reciprocal of corneal sensitivity. Corneal sensitivity can be determined by the corneal touch threshold (CTT) using a Cochet-Bonnet esthesiometer. Five different regions of the cornea can be evaluated (nasal, ventral, lateral, dorsal, and central).

Objective measures for re-epithelialization include slit lamp microscopy with fluorescence staining.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, veterinarian, or researcher taking into account parameters such as physical and physiological factors including target, body weight, severity of wound, type of wound, previous or concurrent therapeutic interventions, idiopathy of the subject, and route of administration.

The amount and concentration of administration form in a composition, as well as the quantity of the pharmaceutical composition administered to a subject, can be selected based on clinically relevant factors, the solubility of the administration form in the composition, the potency and activity of the administration form, and the manner of administration of the composition. A composition including a therapeutically effective amount of an administration form disclosed herein, or a pharmaceutically acceptable salt or prodrug thereof, can be administered to a subject for treatment of wounds in a clinically safe and effective manner, including one or more separate administrations of the composition. For example, about 0.05 mg/kg to about 5.0 mg/kg can be administered to a subject per day in one or more doses (e.g., doses of about 0.05 mg/kg QD, 0.10 mg/kg QD, 0.50 mg/kg QD, 1.0 mg/kg QD, 1.5 mg/kg QD, 2.0 mg/kg QD, 2.5 mg/kg QD, 3.0 mg/kg QD, 0.75 mg/kg BID, 1.5 mg/kg BID or 2.0 mg/kg BID). For certain indications, the total daily dose of administration form can be about 0.05 mg/kg to about 3.0 mg/kg administered to a subject one to three times a day, including administration of total daily doses of about 0.05-3.0, 0.1-3.0, 0.5-3.0, 1.0-3.0, 1.5-3.0, 2.0-3.0, 2.5-3.0, and 0.5-3.0 mg/kg/day of administration forms using 60-minute QD, BID or TID dosing. In one particular example, pharmaceutical compositions can be administered QD or BID to a subject with, e.g., total daily doses of 1.5 mg/kg, 3.0 mg/kg, 4.0 mg/kg of a composition with up to about 92-98% wt/v.

Additional useful doses can often range from 0.1 to 5 μg/kg or from 0.5 to 1 μg/kg. In other examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, or more.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly.

In particular embodiments, the compositions described herein can be used in conjunction with other wound treatments. For example, in the case of a diabetic ulcer, sharp debridement, pressure relief, and various methods of infection control may be used.

In various embodiments, a topical formulation of a composition as described herein can be applied to the wound. In some embodiments, a topical formulation is applied superficially and the wound is then covered by a dressing. In particular embodiments, the dressing is moistened. In some embodiments, the dressing can be moistened by saline. In various embodiments, the dressing can be left in place for up to 6 hours, up to 12 hours, or up to 24 hours. In particular embodiments, the dressing is removed, the topical formulation is reapplied, and a new dressing is used to cover the wound.

The compositions disclosed herein can be administered with additional components to reduce the occurrence of unwanted events during wound healing. For example, the compositions described herein can be administered with therapeutics for the treatment of diabetic ulcers such as Becaplermin (e.g., Regranex® (Smith & Nephew, Inc., Memphis, TN, USA)).

In various embodiments, the compositions described herein can be administered with antiplatelet medications (e.g. irreversible cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, or thromboxane inhibitors), growth factors (e.g. platelet-derived growth factor (PDGF)), and/or vasodilators.

The compositions can also be administered with anti-infective agents including anthelmintics (e.g., mebendazole), antibiotics including aminoclycosides (e.g., gentamicin, neomycin, tobramycin), antifungal antibiotics (e.g., amphotericin b, fluconazole, griseofulvin, itraconazole, ketoconazole, nystatin, micatin, tolnaftate), cephalosporins (e.g., cefaclor, cefazolin, cefotaxime, ceftazidime, ceftriaxone, cefuroxime, cephalexin), betalactam antibiotics (e.g., cefotetan, meropenem), chloramphenicol, macrolides (e.g., azithromycin, clarithromycin, erythromycin), penicillins (e.g., penicillin G sodium salt, amoxicillin, ampicillin, dicloxacillin, nafcillin, piperacillin, ticarcillin), tetracyclines (e.g., doxycycline, minocycline, tetracycline), bacitracin, clindamycin, colistimethate sodium, polymyxin b sulfate, vancomycin, antivirals (e.g., acyclovir, amantadine, didanosine, efavirenz, foscarnet, ganciclovir, indinavir, lamivudine, nelfinavir, ritonavir, saquinavir, stavudine, valacyclovir, valganciclovir, and zidovudine), quinolones (e.g., ciprofloxacin, levofloxacin), sulfonamides (e.g., sulfadiazine, sulfisoxazole), sulfones (e.g., dapsone), furazolidone, metronidazole, pentamidine, sulfanilamidum crystallinum, gatifloxacin, and sulfamethoxazole/trimethoprim.

Compositions can also be administered with anesthetics including ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

### (v) Exemplary Embodiments

1. A method of promoting wound healing and/or treating an ocular condition in a subject including up-regulating in the subject thymosin beta 4 (Tb4) and/or a precursor, active fragment, or active variant thereof; and vasoactive peptide (VIP) and/or a precursor, active fragment, or active variant thereof thereby promoting wound healing and/or treating the ocular condition in the subject.
2. The method of embodiment 1, wherein the up-regulating is through administering a therapeutically effective amount of the thymosin beta 4 (Tb4) and/or precursor, active fragment, or active variant thereof and a therapeutically effective amount of the vasoactive peptide (VIP) and/or precursor, active fragment, or active variant thereof to the subject.
3. The method of embodiment 1 or 2, wherein the precursor, active fragment, or active variant of Tb4 includes SEQ ID NO: 2, 3, or 4, Tb4X, or proTb4.
4. The method of any of embodiments 1-3, wherein the precursor, active fragment, or active variant of VIP includes PreproVIP, ProVIP, N-terminal VIP fragment (1-12), or C-terminal VIP fragment (22-28).
5. The method of any of embodiments 1-4, wherein the subject is a diabetic subject.
6. The method of any of embodiments 2-5, wherein the administering is systemic and/or topical.
7. The method of any of embodiments 2-6, wherein the administering is intradermal, intralesional, intravitreal, intraocular, and/or subcutaneous.
8. The method of any of embodiments 2-7, wherein the administering is through application of a wound dressing.
9. The method of any of embodiments 2-8, wherein the therapeutically effective amounts are administered as multiple doses.
10. The method of embodiment 9, wherein the multiple doses are administered every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly.
11. The method of any of embodiments 1-10, wherein the upregulation occurs with the administration of a second wound treatment.
12. The method of embodiment 10, wherein the second wound treatment includes an antiplatelet medication.
13. The method of embodiment 10, wherein the second wound treatment includes an anti-infective agent.
14. The method of embodiment 10, wherein the second wound treatment includes an anesthetic.
15. The method of any of embodiments 1-14, wherein the promoting wound healing promotes re-epithelialization.
16. The method of any of embodiments 1-15, wherein promoting wound healing and/or treating an ocular condition reduces or delays vision loss, drusen, pigment changes in the retina, abnormal blood vessel growth, leaky blood vessels, macular swelling, corneal swelling, corneal thinning, accumulation of a fatty yellow pigment (lipofuscin), night blindness, distorted vision, blurry vision, rod damage, cone damage, uvea inflammation, eye redness, pain, sensitivity to light, floaters, eye flashes, nodules, orbital inflammation, lacrimal gland enlargement, decreased visual acuity, decreased contrast sensitivity, blind spots, loss of color perception, loss of peripheral vision, fluid build-up in the macula, retinal scarring, double vision, pigment clumps, tunnel vision, thin cornea, spotting, leukocoria, lesions, crystals, or nystagmus,
17. The method of any of embodiments 1-16, wherein the ocular condition includes keratoepitheliopathy, impaired corneal wound healing, or corneal neuropathy.
18. The method of any of embodiments 1-17, wherein the ocular condition includes diabetic retinopathy, diabetic macular edema, corneal ulcer, Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Beliefs disease, Achromatopsia 2, acute posterior multifocal placoid pigment epitheliopathy (APMPPE), acute zonal occult outer retinopathy (AZOOR), adult-onset vitelliform macular dystrophy (AVMD), ocular albinism with late-onset sensorineural deafness (OASD), Alstrom syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline comeoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, or Wagner syndrome.
19. A composition including thymosin beta 4 (Tb4), a precursor, active fragment, and/or active variant thereof; and vasoactive peptide (VIP), a precursor, active fragment, and/or active variant thereof; and a pharmaceutically acceptable carrier.
20. The composition of embodiment 19, wherein the precursor, active fragment, or active variant of Tb4 includes SEQ ID NO: 2, 3, or 4, Tb4X, or proTb4.
21. The composition of embodiment 19 or 20, wherein the precursor, active fragment, or active variant of VIP includes PreproVIP, ProVIP, N-terminal VIP fragment (1-12), or C-terminal VIP fragment (22-28).
22. The composition of any of embodiments 19-21, formulated for systemic, topical, intradermal, intralesional, intravitreal, intraocular, and/or subcutaneous administration.
23. The composition of any of embodiments 19-22, formulated as a gel, ointment, paste, lotion, cream, spray, foam, or powder.
24. The composition of any of embodiments 19-23, formulated for sustained release.
25. The composition of any of embodiments 19-24, incorporated into a wound dressing.
26. The composition of embodiment 25, wherein the wound dressing is a bandage or a transdermal patch.
27. The composition of embodiment 26, wherein the bandage includes an adhesive bandages.
28. The composition of embodiment 27, wherein the adhesive bandage includes a pressure sensitive adhesive.
29. A kit including thymosin beta 4 (Tb4), a precursor, active fragment, and/or active variant thereof; and vasoactive peptide (VIP), a precursor, active fragment, and/or active variant thereof.
30. The kit of embodiment 29, wherein the precursor, active fragment, or active variant of Tb4 includes SEQ ID NO: 2, 3, or 4, Tb4X, or proTb4.
31. The kit of embodiment 29 or 30, wherein the precursor, active fragment, or active variant of VIP includes PreproVIP, ProVIP, N-terminal VIP fragment (1-12), or C-terminal VIP fragment (22-28).
32. The kit of any of embodiments 29-31, further including a pharmaceutically acceptable carrier.
33. The kit of any of embodiments 29-32, further including a wound dressing.
34. The kit of embodiment 33, wherein the wound dressing is a bandage or a transdermal patch.
35. The kit of embodiment 34, wherein the bandage includes an adhesive bandages.
36. The kit of embodiment 35, wherein the adhesive bandage includes a pressure sensitive adhesive.
37. The kit of any of embodiments 29-36, further including a second wound treatment.
38. The kit of embodiment 37, wherein the second wound treatment includes an antiplatelet medication.
39. The kit of embodiment 37, wherein the second wound treatment includes an anti-infective agent.
40. The kit of embodiment 37, wherein the second wound treatment includes an anesthetic.
(vi) Experimental Examples. The Experimental Example below is included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

Experimental Example. Abstract. Despite the prevalence of diabetic retinopathy, the majority of adult diabetic patients develop visually debilitating corneal complications, including impaired wound healing. Unfortunately, there is limited treatment for diabetes-induced corneal damage. This Example investigates a novel, peptide-based combination therapy, thymosin beta-4 and vasoactive intestinal peptide (Tβ4/VIP), against high-glucose-induced damage to the corneal epithelium. Electric cell-substrate impedance sensing (ECIS) was used for real-time monitoring of barrier function and wound healing of human corneal epithelial cells maintained in either normal glucose (5 mM) or high glucose (25 mM)+Tβ4 (0.1%) and VIP (5 nM). Barrier integrity was assessed by resistance, impedance, and capacitance measurements. For the wound healing assay, cell migration was also monitored. Corneal epithelial tight junction proteins (zonula occludens (ZO)-1, ZO-2, occludin, and claudin-1) were assessed to confirm the disclosed findings. Barrier integrity and wound healing were significantly impaired under high-glucose conditions. However, barrier function and cell migration significantly improved with Tβ4/VIP treatment. These findings were supported by high-glucose-induced downregulation of tight junction proteins that were effectively maintained similar to normal levels when treated with Tβ4/VIP. These results strongly support the premise that Tβ4 and VIP work synergistically to protect corneal epithelial cells against hyperglycemia-induced damage. In addition, this work has significant translational impact regarding the treatment of diabetic patients and associated complications of the cornea.

Introduction. Despite the prevalence of diabetic retinopathy, up to 70% of the 460+ million diagnosed diabetics worldwide present with signs of diabetic corneal complications [Schultz R. O., et al. Trans. Am. Ophthalmol. Soc. 1981; 79:180-199; Foundation ID. IDF Diabetes Atlas. 10th ed. Foundation ID; 2021; Kaji Y. Br. J. Ophthalmol. 2005; 89:254-255; Lutty G. A. Investig. Ophthalmol. Vis. Sci. 2013; 54: ORSF81-7]. Collectively, these visually disruptive conditions impact the morphological, metabolic, physiological, and clinical aspects of the cornea. Impaired wound healing is the most widely recognized complication, along with diabetic keratopathy. The latter describes several corneal pathologies resulting from chronic hyperglycemia exposure, such as edema, epithelial defects, recurrent erosions, neuropathy with a loss of corneal sensitivity, and tear film changes, which frequently occur in both type 1 and type 2 diabetes mellitus yet often go undiagnosed in most cases [Ljubimov A. V. Vis. Res. 2017; 139:138-152; Vieira-Potter V. J., et al. Biomed. Res . Int. 2016; 2016:3801570; Cai D., et al. Am. J. Pathol. 2014; 184:2662-2670]. Notably, dysregulated, delayed corneal wound healing increases susceptibility to corneal ulcers, microbial keratitis, and even corneal perforation [Ljubimov A. V. Vis. Res. 2017; 139:138-152; Cai D., et al. Am. J. Pathol. 2014; 184:2662-2670]. However, treatment targeted toward diabetes-induced corneal damage remains limited; thus, further research is warranted to identify potential therapeutic targets.

Barrier integrity of the cornea is largely due to tight junction complexes as they restrict the passage of large molecules and pathogens across the corneal epithelium [Yoshida Y., et al. Investig. Ophthalmol. Vis. Sci. 2009; 50:2103-2108; Van Itallie C. M., Anderson J. M. Semin. Cell Dev. Biol. 2014; 36:157-165; Niessen C. M. J. Investig. Dermatol. 2007; 127:2525-2532; Lee B., et al. J. Immunol. Res. 2018; 2018:2645465; Yi X., et al. Investig. Ophthalmol. Vis. Sci. 2000; 41:4093-4100]. This complex comprises transmembrane proteins, claudins, occludins, junctional adhesion molecules, and tricellulin; meanwhile, ZO-1, ZO-2, and ZO-3 in the peripheral function as scaffolding proteins for intercellular junctions and link transmembrane proteins to the cytoskeleton [Niessen C. M. J. Investig. Dermatol. 2007; 127:2525-2532; Bauer H., et al. J. Biomed. Biotechnol. 2010; 2010:402593]. Compromised epithelial barrier function subsequent to impaired tight junction formation has been indicated as a contributing factor to increased rates of corneal infection in diabetic patients [Jiang Q. W., et al. Acta Pharmacol. Sin. 2019; 40:1205-1211; Chang Y.-S., et al. Sci. Rep. 2020; 10:7388]. Delayed epithelial wound healing; increased corneal sensitivity; dry eye; corneal edema; and, in some cases, corneal neovascularization can also occur as a result of compromised epithelial barrier function in diabetic patients.

Thymosin-β4 (Tβ4) is a highly conserved 43-amino-acid protein endogenously expressed in nearly all tissues except red blood cells [Hannappel E. Beta-Thymosins. Ann. N. Y. Acad. Sci. 2007; 1112:21-37; Erickson-Viitanen S., et al. Arch. Biochem. Biophys. 1983; 221:570-576]. Despite diverse intracellular and extracellular activities, it is widely recognized for its important role in wound healing [Philp D., Kleinman H. K. Ann. N. Y. Acad. Sci. 2010; 1194:81-86]—a therapeutic aspect that is severely lacking in current treatment options for diabetic corneas. In fact, Tβ4 has been found to promote epithelial cell migration in the cornea, inhibit cell apoptosis by modulating inflammatory cytokine/chemokine release, and influence innate immune cell infiltration and function [Ho J. H., et al. Investig. Ophthalmol. Vis. Sci. 2007; 48:27-33; Ho J. H., Su Y., et al. Chin. J. Physiol. 2010; 53:190-195; Sosne G., Kleinman H. K. Investig. Ophthalmol. Vis. Sci. 2015; 56:5110-5117; Carion T. W., et al. Cells. 2018; 7:145; Wang Y., et al. Int. J. Mol. Sci. 2021; 22:11016; Wang Y., et al. Cells. 2021; 10:3579; Zhai Y., et al. Int. J. Mol. Sci. 2022; 23:5458]. Improved wound healing with Tβ4 treatment has been observed in dry eye and alkali burn models [Sosne G., Kleinman H. K. Investig. Ophthalmol. Vis. Sci. 2015; 56:5110-5117; Sosne G., et al. Exp. Eye Res. 2002; 74:293-299] as well, suggesting that it may confer protection in the diabetic cornea. Moreover, reduced Tβ4 levels have been reported in the cornea of diabetic patients with proliferative diabetic retinopathy [Saghizadeh M., et al. Investig. Ophthalmol. Vis. Sci. 2005; 46:3604-3615].

Vasoactive intestinal peptide (VIP) is an endogenous peptide that is produced by neurons and immune cells to function, in part, as an immunomodulator. The potential effects of VIP are not solely due to diabetes-induced enteric nervous system dysfunction, but it has the capacity to function in a more global manner given its widespread distribution [Said S. I., Mutt V. Science. 1970; 169:1217-1218; Said S. I., Rosenberg R. N. Science. 1976; 192:907-908; Henning R. J., Sawmiller D. R. Cardiovasc. Res. 2001; 49:27-37; Szliter E. A., et al. J. Immunol. 2007; 178:1105-1114]. In this regard, previous reports have shown that VIP is protective against high-glucose-induced blood-retinal barrier permeability [Maugeri G., et al. J. Cell. Physiol. 2017; 232:1079-1085; Scuderi S., et al. Peptides. 2013; 39:119-124], and it was found to decrease vascular endothelial growth factor (VEGF) expression in retinal pigment epithelial cells in an in vitro model of diabetic macular edema [Maugeri G., et al. J. Cell. Physiol. 2017; 232:1209-1215]. In addition, VIP treatment of human retinal endothelial cells significantly reduced high-glucose-induced tumor necrosis factor (TNF)-α and VEGF levels while increasing the pro-resolving lipid mediator Resolvin D1 (RvD1) and its receptor G protein-coupled receptor (GPR) 32 [Shi H., et al. Prostagland. Other Lipid Mediat. 2016; 123:28-32]. Activation of pro-resolving circuits such as RvD1 and GPR32 is likely to be beneficial in the context of the diabetic cornea for several reasons, including the resolution of inflammation, and enhanced wound healing and tissue repair.

Both Tβ4 and VIP promote tissue wound healing and regulate inflammation; however, whether they synergistically influence the function of the corneal epithelium under hyperglycemic conditions has yet to be investigated. In this Example, Tβ4 and VIP were examined as a novel, peptide-based combination therapy to prevent high-glucose-induced impairment of corneal epithelial cell barrier function and migration.

### Materials and Methods

Cell Culture of Human Telomerase-Immortalized Corneal Epithelial Cell Line. Human telomerase-immortalized corneal epithelial cells (HUCLs), kindly provided by Dr. Fu-Shin Yu (Wayne State University, Detroit, MI, USA), were previously generated using a retroviral vector that encodes for the human telomerase reverse transcriptase, resulting in immortalization of the cell line [Zhang J., et al. Investig. Ophthalmol. Vis. Sci. 2003; 44:4247-4254]. Cells were cultured in Dulbecco's Modified Eagle Medium (DMEM)/Nutrient Mixture F-12 (F-12) (Thermo Scientific, Wyman, MA, USA) supplemented with 10% fetal bovine serum (FBS) (Atlantic Biological, Norcross, GA, USA) and 1% penicillin/streptomycin, and maintained in a humidified 5% CO₂ incubator at 37° C. HUCLs were used between passages 3 and 5 for all experiments and were authenticated and confirmed to be free from mycoplasma prior to experimentation.

Cell seeding densities were as follows: 60,000 cells/well for ECIS experiments; 300,000 cells/6-well plate for protein analysis; and 300,000 cells/6-well plate containing coverslips coated with fibronectin collagen (FNC Coating Mix, Athena Environmental Service, Inc., Baltimore, MD, USA) for immunostaining. Experimental conditions consisted of normal glucose (5 mM; NG) and high glucose (25 mM; HG)+Tβ4 (Regenerx Biopharmaceuticals Inc., Rockville, MD, USA), and VIP (Bachem, Torrance, CA, USA). Tβ4 and VIP treatments were administered twice per day to achieve final concentrations of 0.1% and 5 nM, respectively. Mannitol (20 mM) was used as an osmotic control.

Conducting ECIS Experiments for Barrier Function and Cell Migration. Barrier function and cell migration were determined using the ECIS Ze system (Applied Biophysics Inc., Troy, NY, USA). Each array (Applied Biophysics Inc.) consists of a 96-well plate, with each well containing either an electrode with an inter-digitated finger configuration (total electrode area=3.92 mm2) for barrier function (array 96W20idf) or two circular 350 μm diameter electrodes (total electrode area=0.256 mm2) for cell migration (array 96W1E+). The arrays were pre-treated as previously described [Ebrahim A. S., et al. Sci. Rep. 2022; 12:14126]. Before inoculation, electrode impedance values were stabilized to reduce electrode drift. After seeding, the HUCLs were switched to serum-free media 12-16 hours (h) prior to initiating experimental conditions.

Plates were maintained at a constant current of 1 μA. The runs were carried out under multiple frequencies (1000 Hz-64 kHz) and monitored continuously with measurements obtained 2 min apart. Barrier function assessments were carried out for 120 h. For cell migration, an automated wound was induced (wound time—60 s, current—2800 μA, frequency—60 kHz), producing two cell-free areas within the cell monolayer. Cell migration assessments were carried out for a duration of 70 h.

Data Analysis and Modeling. ECIS measurements were obtained as previously reported [Ebrahim A. S., et al. Sci. Rep. 2022; 12:14126] for overall resistance (4 kHz), impedance (32 kHz), and capacitance (64 kHz) as a function of time. Cell data were normalized by comparison with cell-free electrodes. In addition, impedance (y-axis) was measured as a function of frequency (x-axis) and time (z-axis), and represented as a 3D plot.

Mathematical modeling was applied to derive three parameters: Rb (resistance between cells, Ω-cm2), α (basolateral resistance between cells and underlying substrate, Ω-cm1/2), and cm (cell membrane capacitance, μF/cm²). To validate the modeled data, Drift Correction and Model Fit RMSE (root mean square error) values were used. The migration velocity in response to wounding was calculated by dividing the total distance that the cells migrated over the electrode radius (175 μm) by the time required to fully recover the normalized resistance prior to wounding.

Western Blot Analysis. After six days of experimental conditions, cellular lysates were prepared using RIPA buffer (Cell Signaling Technology, Danvers, MA, USA) containing phosphatase and protease inhibitors (Thermo Fisher Scientific, Waltham, MA, USA), then centrifuged at 14,000× gravity (g) for 15 min. Supernatants were further normalized to ensure equal amounts of protein using the bicinchoninic acid (BCA) method (Thermo Scientific). Subsequently, samples were separated onto 4-20% tris-glycine gels (Invitrogen, Carlsbad, CA, USA) and transferred to polyvinylidene fluoride (PVDF) membranes. Next, membranes were blocked using 5% non-fat milk dissolved in TBST (10 mM Tris-HCl buffer, pH 8.0, 150 mM NaCl, and 0.1% Tween 20) at room temperature for 30 min. Membranes were then incubated at 4° C. overnight with antigen-specific primary rabbit antibodies as follows: anti-ZO-1 (1:500; Cat #40-2200, Invitrogen, Waltham, MA, USA), anti-ZO-2 (1:500; Cat #2847), anti-occludin (1:500; E6B4R, Cat #91131), anti-claudin-1 (1:500; Cat #4933) (Cell Signaling Technology, Danvers, MA, USA), and anti-β-actin (1:1000; C4; Cat #sc-47778; Santa Cruz Biotechnology, Dallas, TX, USA). Each blot was incubated with goat anti-rabbit HRP-conjugated secondary antibodies (Cat #111-035-144; Thermo Fisher Scientific) for 1 h at room temperature, followed by enhanced chemiluminescence (ECL) Chemiluminescent Substrate incubation (Thermo Fisher Scientific). After detection of ZO-1, ZO-2, occludin, and claudin-1, Restore PLUS Western blot stripping buffer (Thermo Fisher) was used to strip PVDF membranes (15 min at room temperature), which were then re-probed for β-actin (internal control). Western blot images were collected (Bio-Rad Molecular Imager, ChemiDoc XRS+; Azure Imaging System A1600-1360) and analyzed using Image Studio Lite software version 5.2 (LI-COR Biosciences, Lincoln, NE, USA). Target protein expression was quantified after normalizing to β-actin.

Immunostaining of Tight Junction Molecules. Barrier integrity of the epithelium was qualitatively evaluated through ZO-1 and occludin immunostaining. Changes in cellular proliferation were determined by Ki-67 immunostaining. After five days, cells were confirmed to be confluent before coverslips were fixed (Z-Fix; Anatech Ltd., Battle Creek, MI, USA) for 15 min at room temperature. Next, cells were permeabilized (0.05% triton X-100, 15 min on ice) and rinsed (phosphate-buffered saline (PBS), 2×), followed by blocking (5% bovine serum albumin (BSA), 1 h). Cells underwent overnight incubation at 4° C. with anti-ZO-1 rabbit monoclonal antibody (mAb) Alexa Fluor 488 Conjugate (1:100) (D6L1E; Cat #86942; Cell Signaling Technologies), fluorescein isothiocyanate (FITC)-conjugated anti-occludin mouse mAb (1:200) (OC-3F10; Cat #33-1511; Invitrogen, Eugene, OR, USA), or FITC-conjugated anti-Ki-67 mouse mAb (1:100) (Cat #556026; BD Biosciences, San Diego, CA, USA). Each coverslip was rinsed and mounted using Prolong Diamond plus 4′,6-diamidino-2-phenylindole (DAPI) (Invitrogen, Waltham, MA, USA). Final images were taken with an Olympus DP72 microscope with a 40× objective lens UApo N 340 40×/1.35 Oil. Using cellSens dimensions software (cellSens Dimension_1_18), images were captured with a 360 nm filter set to view DAPI (blue) and 488 nm filter set to view FITC (green). Images for green and blue channels in each group were overlapped using ImageJ 1.44 software (NIH, Bethesda, MD, USA).

Statistical Analysis. ECIS assays were conducted in five independent experiments, and Western blot and immunocytochemistry (ICC) assessments from at least three independent experiments. ECIS data are presented as mean±standard error of the mean (SEM), and Western blot data are presented as mean±SD. All data were analyzed by a one-way analysis of variance (ANOVA) followed by Bonferroni's multiple comparison test (GraphPad Prism; San Diego, CA, USA). Data were considered statistically significant at p≤0.05. Unless indicated differently, the data shown are representative of a typical experiment. Group sizes, determined prior to experimentation, are indicated in the figure descriptions.

### Results.

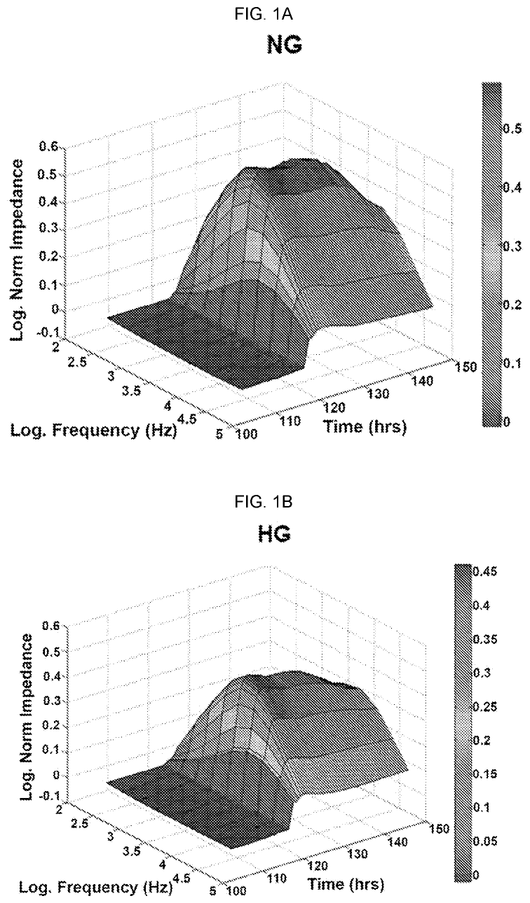

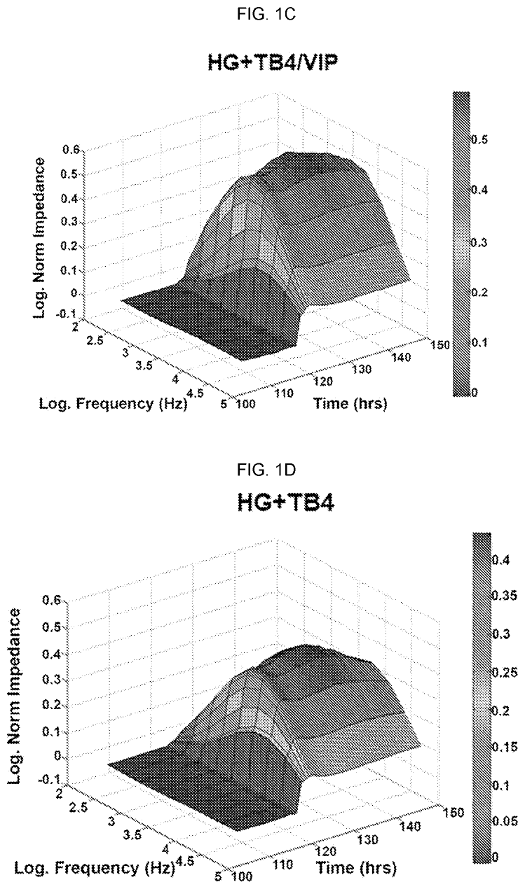

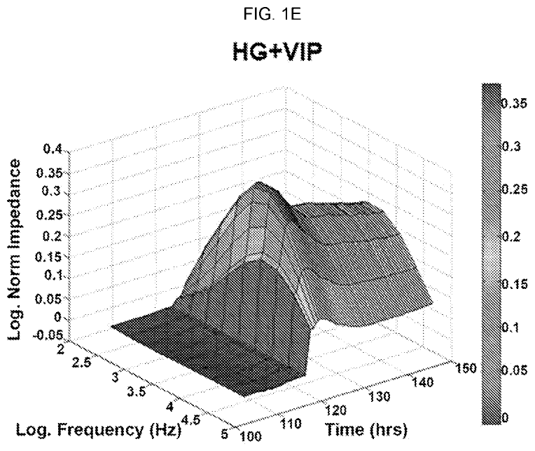

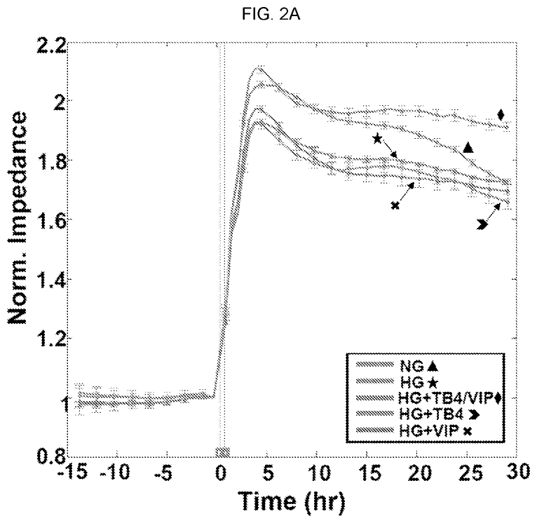

Bioimpedance Analysis of Barrier Function. The influence of hyperglycemic conditions and peptide treatment was assessed by bioimpedance analysis, as shown in FIGS. 1A-1E. The formation of a mature confluent barrier is demonstrated by the plateau in impedance, shown as log-normalized values on the y-axis. Cells maintained in NG reached a higher impedance value (0.4Ω) compared with HG (0.3Ω). Of the different treatment groups exposed to HG, only the combination therapy Tβ4/VIP (0.45Ω) maintained impedance values similar to NG. On the other hand, Tβ4 and VIP monotherapies (0.3 and 0.25Ω, respectively) remained comparable to HG conditions.

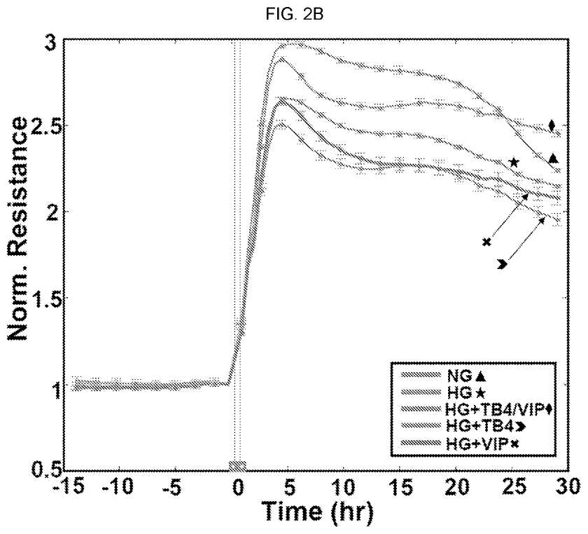

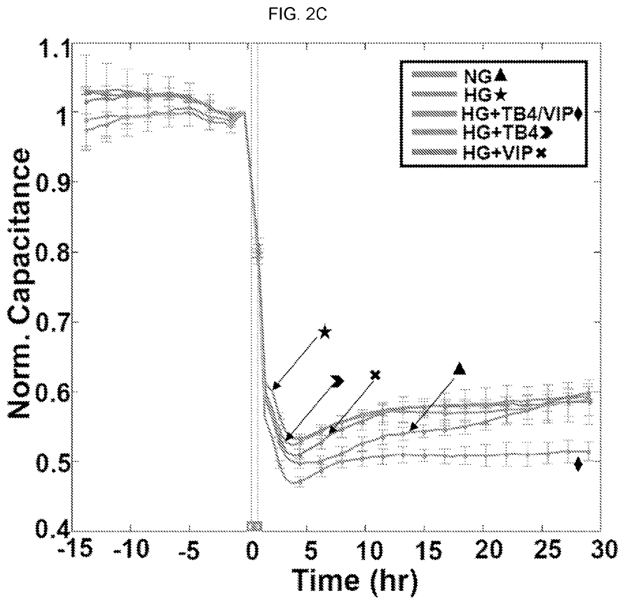

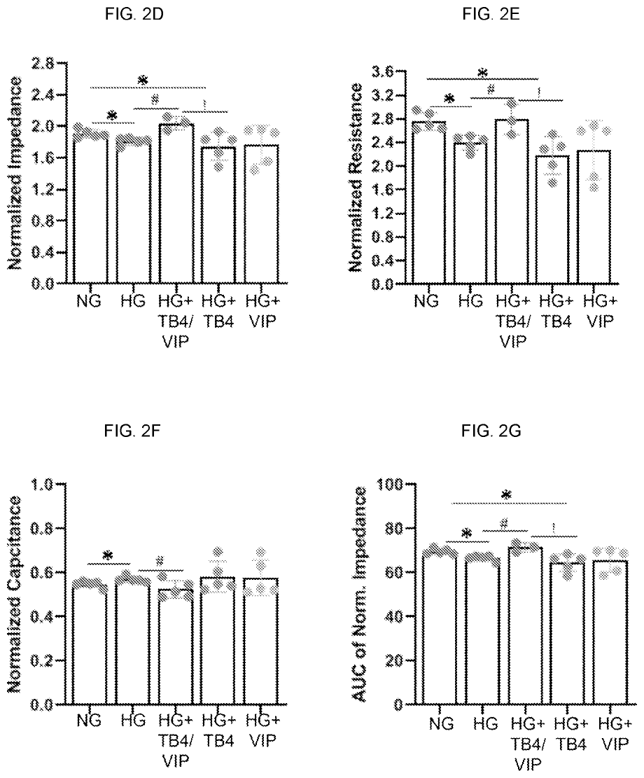

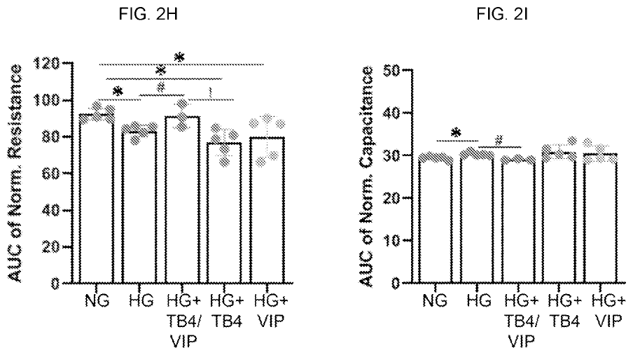

Barrier Function Measurements. Impedance, resistance, and capacitance plot tracings are shown in FIGS. 2A-2C, respectively. HUCLs maintained in HG displayed significantly lower Impedance (FIGS. 2D, 2G), resistance (FIGS. 2E, 2H) and capacitance (FIGS. 2F, 2I) are shown as endpoint and area under the curve (AUC) values. HUCLs maintained in HG displayed significantly lower impedance and resistance and significantly higher capacitance when compared with cells in NG, indicating that the corneal epithelial barrier under NG conditions is not as tight or strong comparatively.

In addition, only Tβ4/VIP-treated HUCLs prevented the HG-induced decreases in both impedance (FIGS. 2D, 2G) and resistance (FIGS. 2E, 2H). No effect, however, was observed regarding either parameter after Tβ4 or VIP monotherapy when exposed to HG. Similarly, only Tβ4/VIP-treated HUCLs mitigated HG-induced increases in capacitance (FIGS. 2F, 2I) values, while no differences were observed with either monotherapy. Taken together, only the combination Tβ4/VIP therapy was able to maintain the characteristics of these cells despite HG conditions.

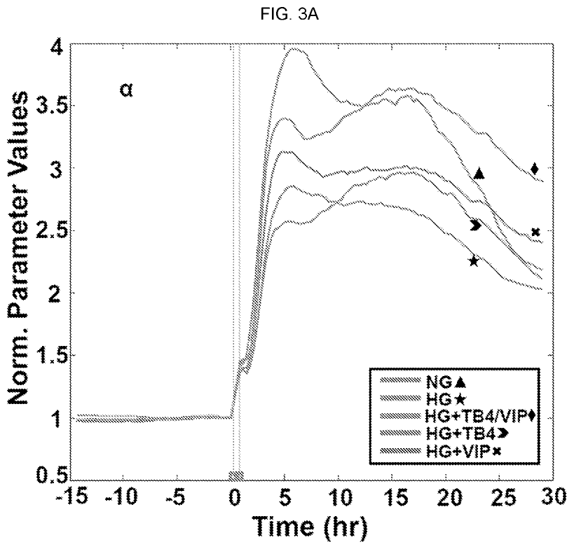

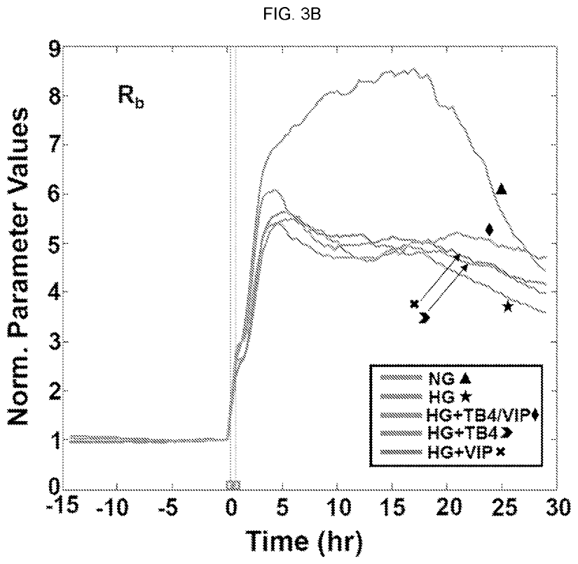

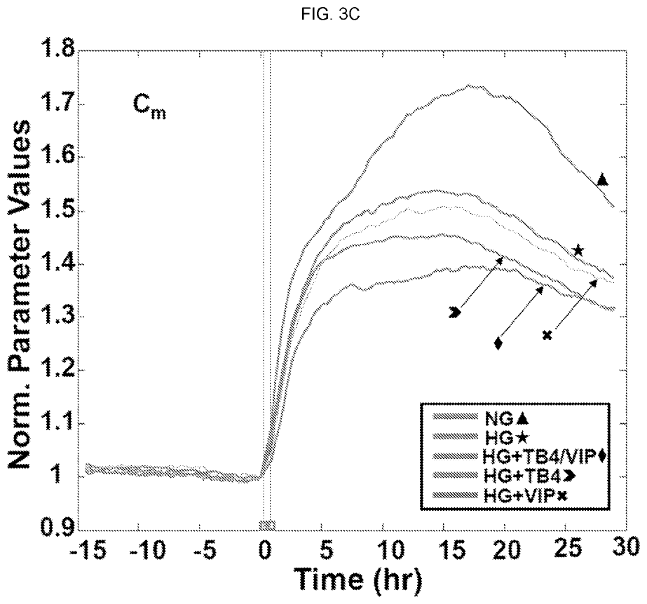

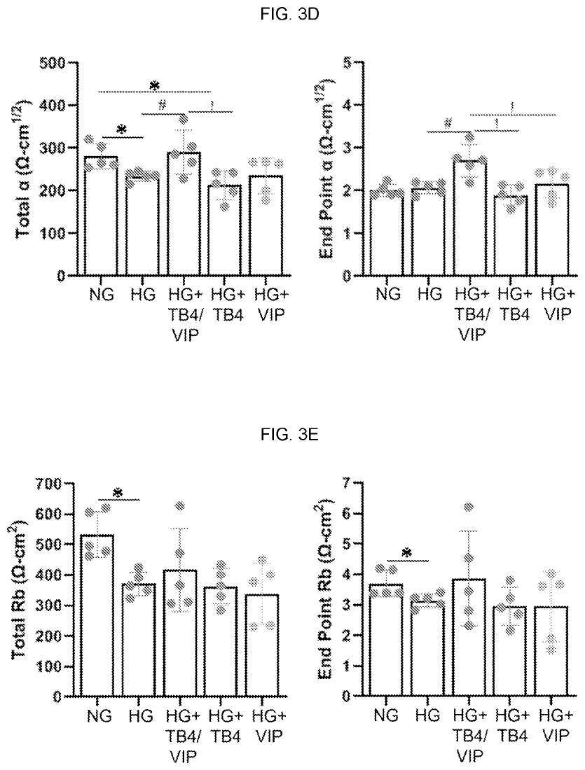

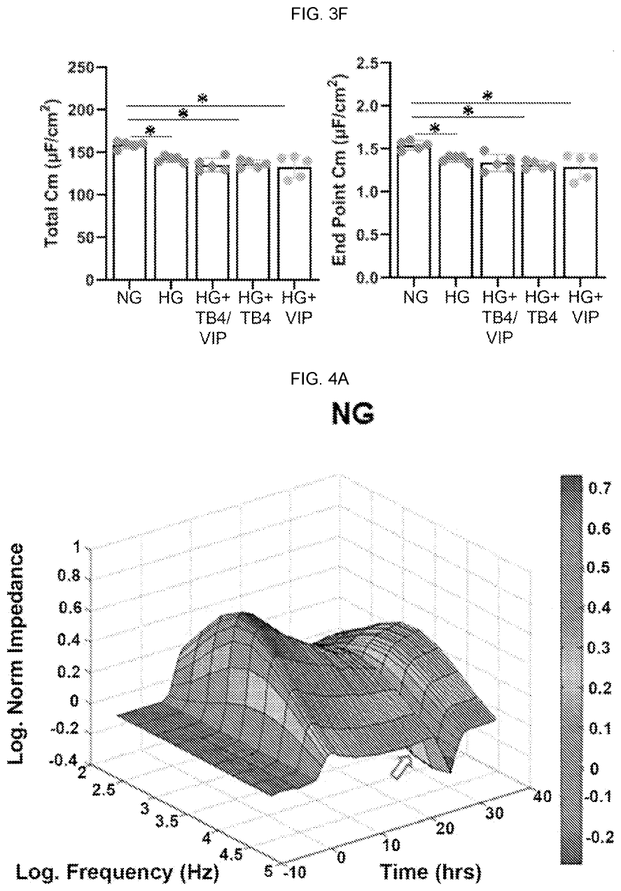

Mathematical Modeling Parameters of the R Data—α, Rb, and Cm. One of the benefits of the ECIS software is the ability to model the impedance (Z) into parameters that distinguish between cell-cell (Rb) and cell-matrix (a) adhesions, as well as membrane capacitance (Cm). Rb, a, and Cm were extrapolated from the generated impedance data and are presented as plot tracings in FIGS. 3A-3C, respectively. Both total and endpoint values for Rb (FIG. 3D), indicative of paracellular barrier strength, were significantly decreased in HUCLs maintained in HG compared with NG conditions. Total a, indicating the strength of interaction between the HUCLs and the basal substrate, was also significantly lower with HG exposure (FIG. 3E), despite no difference in endpoint values. Further, both total and endpoint Cm values (FIG. 3F), used to determine if changes in the capacitance are a result of changes in electrode coverage or are a function of microvariations in the apical membrane structures, were also significantly decreased in cells maintained in HG compared with NG.

Only the combination Tβ4/VIP treatment resulted in a significant increase in a values (both total and endpoint) compared with HG (FIG. 3D), whereas no differences were detected for Rb or Cm (FIGS. 3E and 3F, respectively). No differences were observed for either monotherapy compared with HG. Thus, these data indicate that Tβ4/VIP treatment results in stronger cell-matrix interactions despite HG conditions.

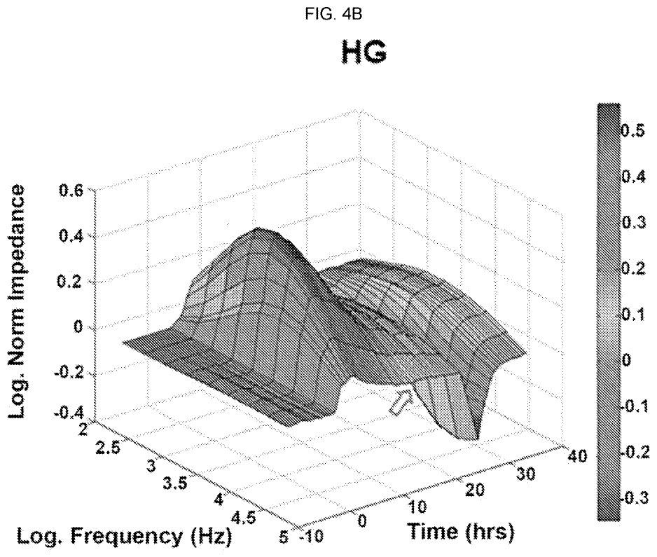

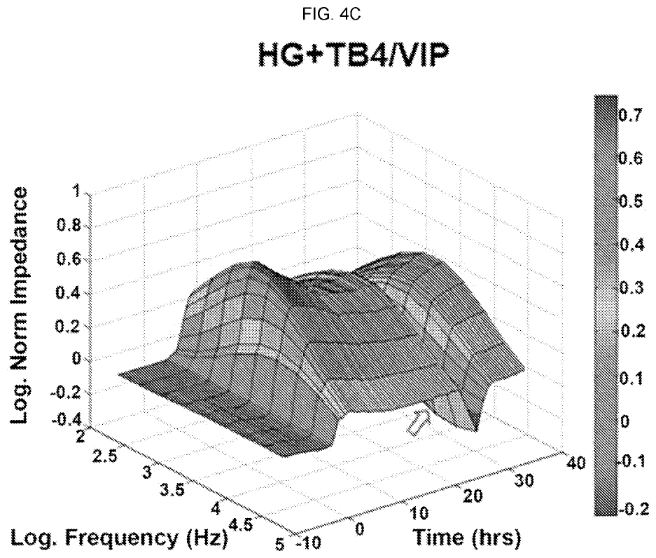

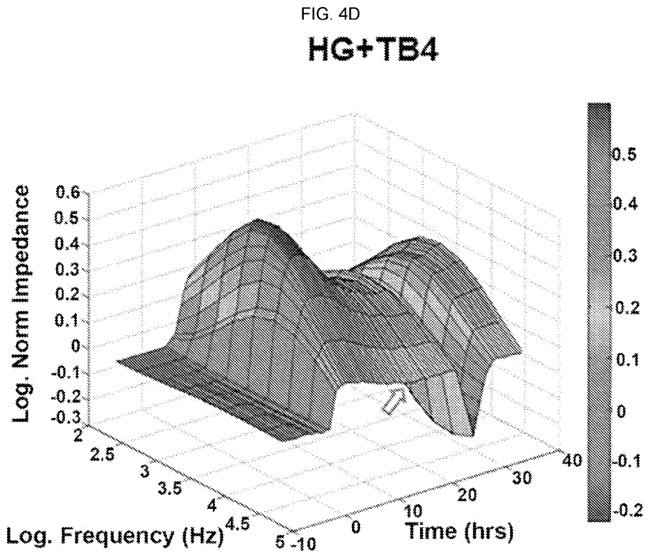

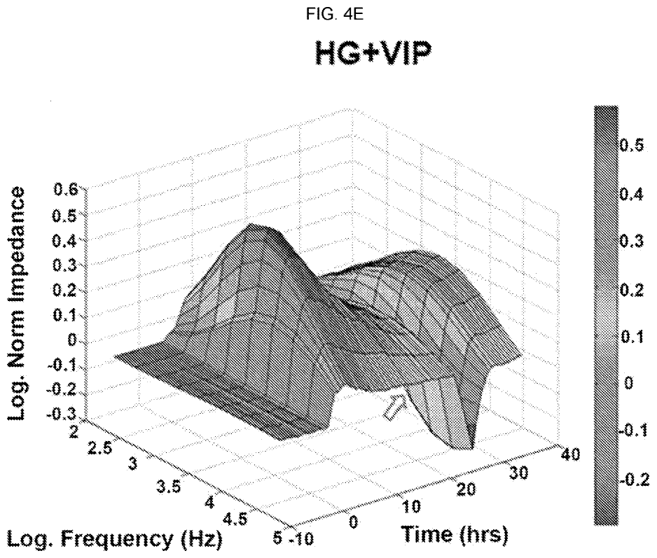

Bioimpedance Analysis of Wound Healing Response. The wound healing response was next assessed by ECIS to further investigate the efficacy of Tβ4/VIP treatment. FIGS. 4A-4E shows the 3D bioimpedance analysis of cell migration and wounding for NG and HG+Tβ4 and VIP. Once a functional barrier was in place (plateau), a high field pulse was induced (red arrow), producing a wound, represented by the drop (valley) in normalized impedance. HUCLs maintained in NG reestablished a stronger barrier after wounding compared with those in HG, as evidenced by the log-normalized impedance values of 0.3Ω for NG (FIG. 4A) and 0.1Ω for HG (FIG. 4B). HUCLs maintained in HG+Tβ4/VIP (FIG. 4C) recovered to 0.4Ω, greater than that of NG and both Tβ4 (0.3Ω) (FIG. 4D) and VIP (0.25Ω) (FIG. 4E) monotherapies.

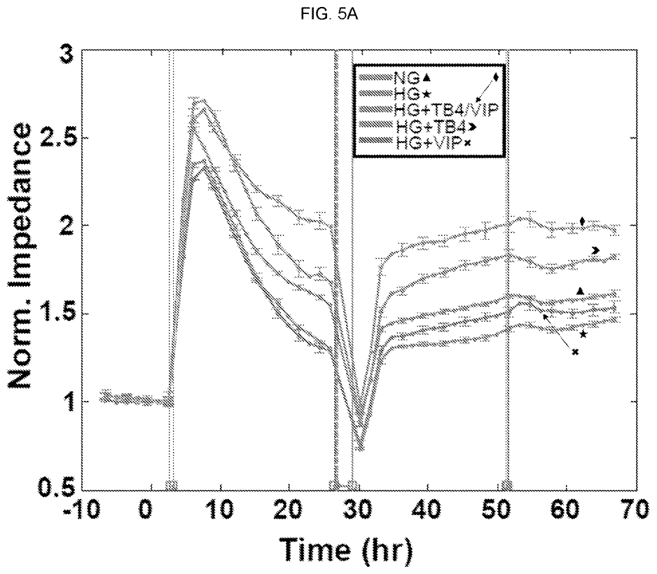

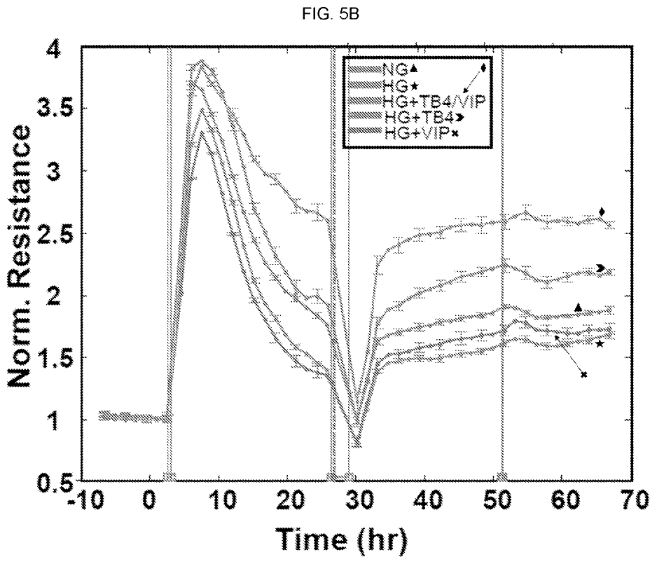

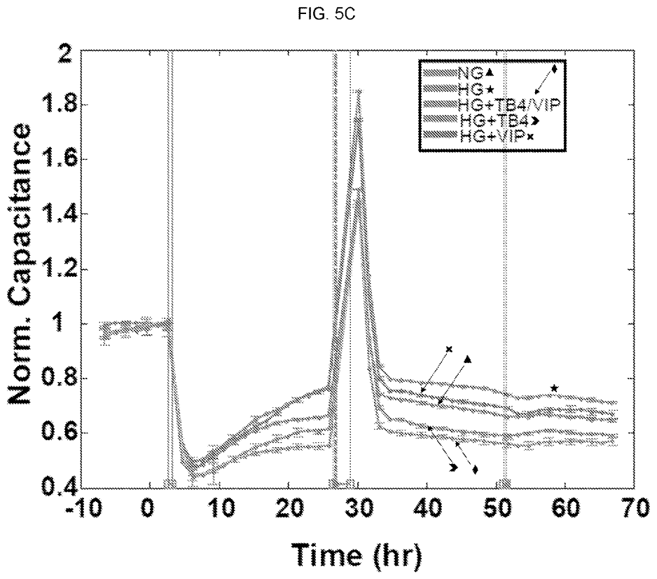

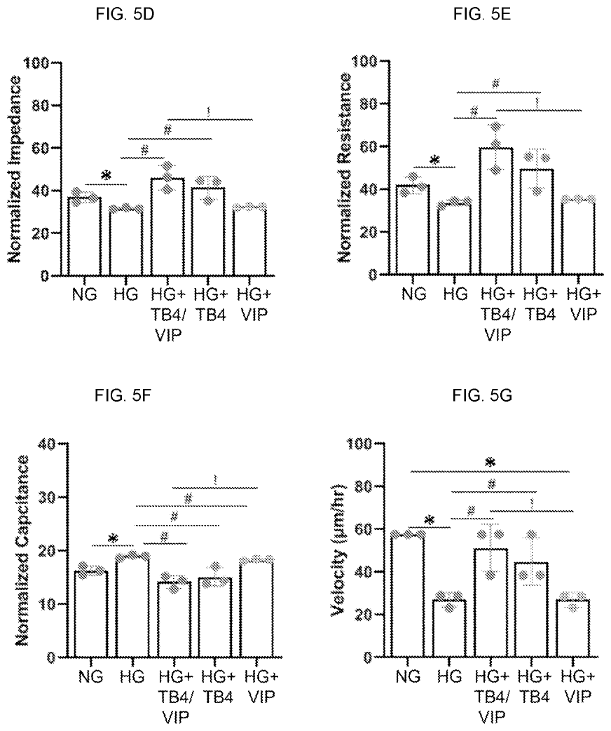

Wound Healing Measurements. To further assess the wound healing response of HUCLs maintained in HG and the therapeutic effects of the combination Tβ4/VIP treatment, normalized impedance, resistance, capacitance, and wound velocity were determined (FIGS. 5A-5G). HG conditions significantly reduced both impedance (FIGS. 5A, 5D) and resistance (FIGS. 5B, 5E) after wounding compared with NG conditions, while capacitance (FIGS. 5C, 5F) was significantly elevated. In addition, the cell velocity (FIG. 5G) was significantly reduced following wounding of HUCLs maintained in HG compared with those in NG.

Similar to the effect observed with barrier function, the combination of Tβ4/VIP significantly increased impedance, resistance, and cell velocity; capacitance was significantly decreased despite the HG conditions. Given the powerful wound healing properties associated with Tβ4, it was not surprising that the Tβ4 monotherapy also significantly elevated impedance, resistance, and cell velocity, along with significantly decreasing capacitance. No differences were observed between impedance, resistance, capacitance, or cell velocity for the VIP monotherapy compared with HG; however, capacitance was significantly reduced.

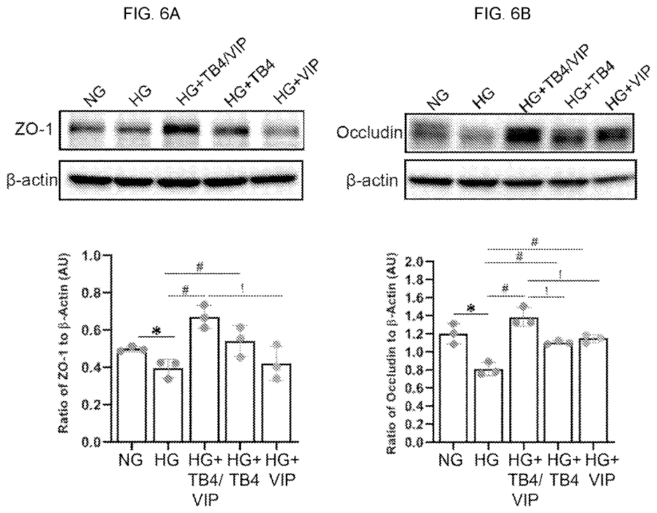

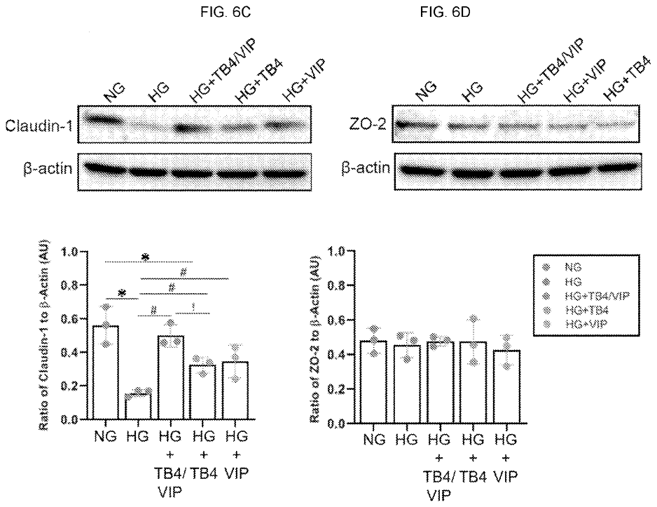

Western Blot Analysis of the Tight Junction Protein Complexes. As an extension of the ECIS study, alterations in tight junction proteins ZO-1, ZO-2, claudin-1, and occludin were also determined. As shown in FIGS. 6A-6D, protein levels for ZO-1 (FIG. 6A), occludin (FIG. 6B), and claudin-1 (FIG. 6C) were significantly reduced in HUCLs maintained under HG compared with those under NG conditions. Remarkably, Tβ4/VIP significantly upregulated all three molecules compared with HG-treated cells. Somewhat unexpectedly, the Tβ4 monotherapy also resulted in a significant increase in ZO-1, occludin, and claudin-1, while the VIP monotherapy significantly upregulated levels of occludin and claudin-1 compared with HG. No effects were observed between any of the groups regarding ZO-2. Taken together, these results reveal that Tβ4/VIP treatment is a more efficacious therapy than either single-peptide monotherapy, which strongly suggests that Tβ4 works synergistically with VIP to enhance the observed functional effect.

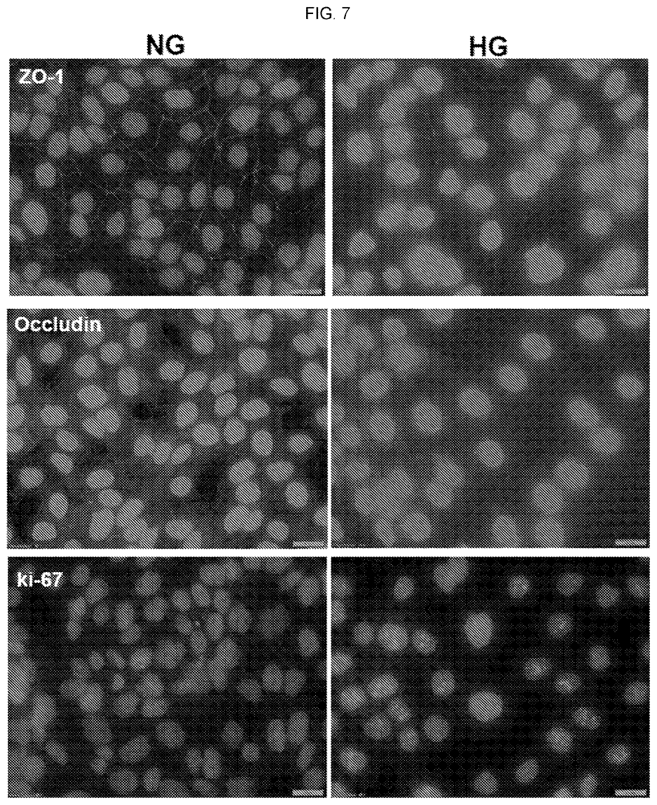

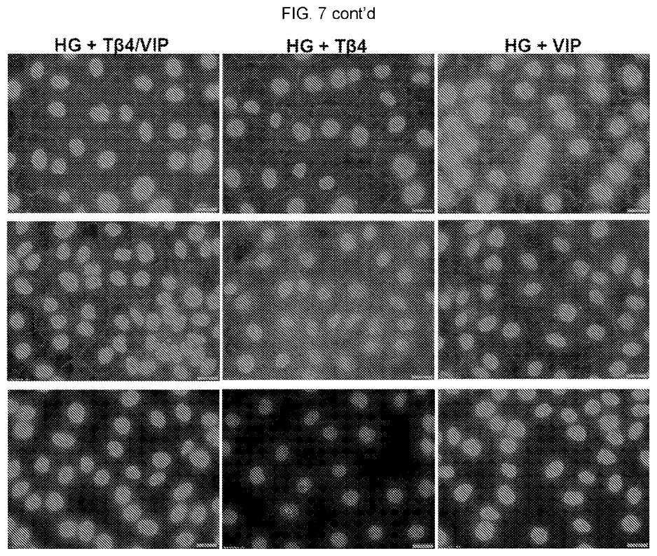

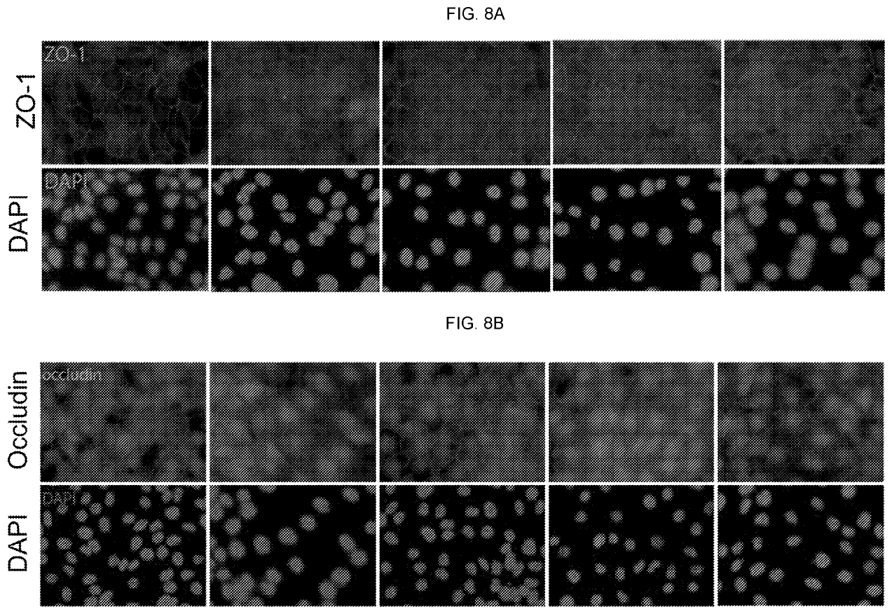

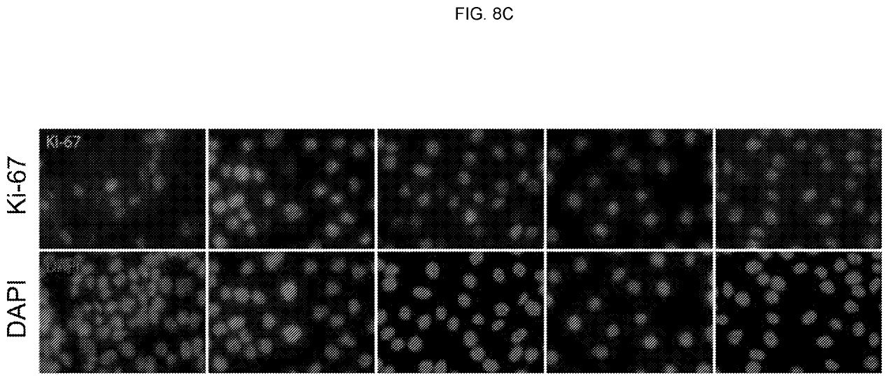

IHC Assessment of the Tight Junction Protein Complexes. IHC was carried out to detect ZO-1 and occludin expression in HUCLs maintained in either NG or HG+Tβ4/VIP treatments (FIG. 7). Under NG conditions, ZO-1 staining (top row) presented specifically along the border of each cell, highlighting the cobblestone-like appearance of epithelial cells. There were no observable gaps or indications of membrane swelling. Occludin staining (middle row) displayed a similar pattern under NG conditions. There was positive staining along the plasma membrane, reflecting that occludin is localized to the intercellular junctional complexes. While it is mainly localized at tight junctions, some positive staining was observed intracellularly. Both ZO-1 and occludin staining intensities were lower in HUCLs maintained in HG compared with those in NG. The continuous linear pattern of ZO-1 and occludin staining associated with intercellular junctional complexes appears to be markedly reduced with HG exposure. Single channel images are illustrated in FIGS. 8A-8C.

Additionally, HUCLs maintained in HG appeared to be more flattened or elongated compared with the normal cobblestone-like morphology, with fewer cell-cell interactions, as reflected by ZO-1 and occludin staining. Although Tβ4 and VIP monotherapies improved the intensity of both ZO-1 and occludin staining, the cell morphology remained somewhat flattened and elongated, whereas combination Tβ4/VIP treatment of HUCLs maintained under HG conditions resulted in stronger staining of ZO-1 as a distinct band-like pattern at the cell borders with more punctate staining outlining the boundaries between adjacent cells. Occludin staining was more intense, not only reflecting a similar band-like pattern along cell borders, but also more positive staining intracellularly, likely reflecting the trafficking dynamics and cellular processes influenced by HG conditions.

HUCL Cell Proliferation. HUCLs were also stained for the proliferation marker Ki-67 (FIG. 7, bottom row) to determine whether the observed changes were due, in part, to differences in cellular proliferation rates. Positive Ki-67 staining was observed as nuclear with a punctate appearance in all groups. However, HUCLs maintained in HG appeared to have more Ki67+ cells and more intense staining when compared with those in NG, while the Tβ4/VIP, Tβ4, and VIP treatment groups demonstrated staining patterns comparable to NG despite HG exposure, indicating similar proliferation rates between the three treatment groups and the NG controls.

Discussion Diabetic corneal complications, including impaired epithelial barrier function, delayed wound healing, and increased susceptibility to infection, pose significant challenges in the management of diabetic patients. These complications can lead to vision impairment, severely impacting the quality of life for individuals with diabetes. Despite advances in diabetes management, there remains a critical need for effective treatments targeting corneal complications. In this Example, the therapeutic efficacy of Tβ4/VIP in mitigating diabetic corneal complications was investigated by evaluating its effects on corneal epithelial barrier function and wound healing.

Previous work has illustrated that Tβ4 influences multiple wound healing pathways in the cornea, including fibronectin: integrin and uPA:uPAR [Carion T. W., et al. Cells. 2018; 7:145]. In the clinical setting, Tβ4 treatment has been shown to reduce non-healing epithelial defects in diabetic patients with neurotrophic keratopathy [Dunn S. P., et al. Ann. N. Y. Acad. Sci. 2010; 1194:199-206]. Previous work in a bacterial keratitis model also revealed that VIP treatment modulates cytokine/chemokine production, decreases inflammatory cell infiltration, and enhances growth factor production and β-defensins levels, subsequently promoting healing and tissue restoration in the infected cornea [Szliter E. A., et al. J. Immunol. 2007; 178:1105-1114; Berger E. A., et al. Investig. Ophthalmol. Vis. Sci. 2010; 51:5776-5782; Jiang X., et al. Investig. Ophthalmol. Vis. Sci. 2011; 52:6154-6161]. The Yu lab linked defects in corneal epithelial cell-dendritic cell-sensory nerve interactions to the pathogenesis of neurotrophic keratopathy [Yu F. S., et al. Expert. Rev. Ophthalmol. 2015; 10:383-392; Gao N., et al. J. Clin. Investig. 2016; 126:1998-2011; Zhang Y., et al. Diabetes. 2020; 69:1549-1561], while also showing that VIP partially restored corneal epithelial wounds after debridement in an STZ-induced T1DM mouse model [Villarroel M., et al. Exp. Eye Res. 2009; 89:913-920]. Although Tβ4 and VIP have been investigated as monotherapies in the eye, it is unknown whether the two molecules can work synergistically to further improve the pathogenic hyperglycemia-induced effects on corneal epithelial cells.

Use of ECIS biosensor technology allows insight into key aspects of corneal epithelial cellular behavior in response to HG exposure and how this response is altered in the presence of Tβ4/VIP combination treatment while also replacing the use of animals in research. Impedance is the overall opposition to the AC flow. Monitoring changes in impedance over time provides insights into various cellular processes, such as cell adhesion, spreading, proliferation, migration, and barrier integrity. Impedance measurements by ECIS reflect alterations in cell morphology, attachment, and interactions with the substrate. The observed decrease in impedance in cells maintained in HG indicates cell detachment or loss of cell-substrate contacts, whereas increased impedance with Tβ4/VIP treatment signifies that cell spreading, proliferation, or the formation of cell-cell contacts have been maintained despite HG exposure.

Impedance is a value that consists of both resistance and capacitance. Resistance represents the opposition to the flow of electrical current and is a measure of the integrity and tightness of the corneal epithelial barrier. It reflects resistance to the passage of ions and molecules through the paracellular pathway between adjacent cells. The higher resistance values observed under NG conditions indicate a more intact and tightly sealed barrier, with reduced paracellular permeability. The reduced resistance values obtained from HG conditions suggest reduced cell-substrate adhesion, loss of barrier integrity with increased paracellular permeability, cell detachment or loss of viability, and altered cellular morphology. These data coincide with previous findings that HG conditions reduce the barrier integrity of corneal epithelial cells [Jiang Q. W., et al. Acta Pharmacol. Sin. 2019; 40:1205-1211], retinal pigment epithelial cells [Villarroel M., et al. Exp. Eye Res. 2009; 89:913-920], and intestinal epithelial cells [Villarroel M., et al. Exp. Eye Res. 2009; 89:913-920; Thaiss C. A., et al. Science. 2018; 359:1376-1383]. Remarkably, Tβ4/VIP prevented these HG-induced changes in the barrier integrity and function of human corneal epithelial cells.

Lower membrane capacitance values, as observed in HUCLs maintained under NG conditions, reflect tighter and more stable cell-cell contacts at tight junctions. They also indicate increased electrical coupling between adjacent corneal epithelial cells. Although compromised when cells were maintained under HG conditions, Tβ4/VIP effectively prevented these pathogenic changes. As a result, cell-cell communication is maintained, facilitating coordinated cellular responses, such as the synchronization of cell behavior and signaling within the epithelium, which can be important for maintaining tissue function and integrity.

Mathematical modeling revealed that HG lowered Rb—indicating alterations in cell-substrate interactions and changes in cell morphology; lowered α—suggesting changes in cell adhesion, spreading, and migration or changes in cell-substrate interactions; and increased Cm—a reflection of morphological changes, alterations in membrane dynamics, or changes in the protrusions or extensions of the cell surface. Tβ4/VIP effectively strengthened adhesion between the cells and the substrate, indicating better cell attachment and spreading; however, cell-substrate interactions and changes in cellular morphology likely contributed to the overall differences observed in the resistance data.

Regarding the wound healing process, as the corneal epithelial cells migrate and cover the cell-free region, impedance gradually increases, reflecting the restoration of cell-substrate adhesion, reformation of tight junctions, and reestablishment of the epithelial barrier. This is observed in cells maintained in NG, and was markedly impaired in HUCLs exposed to HG, as indicated by the impedance, resistance, capacitance, and cell velocity changes. These results correlate with a previous in vivo study showing that cells in high glucose have weaker electric fields and migrate slower [Shen Y., et al. Sci. Rep. 2016; 6:26525]. Remarkably though, the impaired response under HG conditions was fully mitigated in cells exposed to HG+Tβ4/VIP. These data demonstrate that Tβ4/VIP effectively facilitates the migration and proliferation of corneal epithelial cells during the healing process, contributing to efficient closure of the wound and restoration of corneal integrity even under HG conditions.

The assessment of tight junction complexes is integral to understanding the function and dynamics of corneal epithelial cells. The assembly of tight junction protein molecules, such as occludin, claudins, and ZO-1, plays a crucial role in maintaining the integrity and selective permeability of the corneal epithelial barrier [Van Itallie C. M., Anderson J. M. Semin. Cell Dev. Biol. 2014; 36:157-165; Niessen C. M. J. Investig. Dermatol. 2007; 127:2525-2532; Lee B., et al. J. Immunol. Res. 2018; 2018:2645465]. ZO-1 is a cytoplasmic protein that acts as a scaffolding protein, linking transmembrane tight junction proteins, such as occludin and claudins, to the actin cytoskeleton. The selection of claudin-1 was based on previous reports demonstrating that it was expressed in corneal and conjunctival epithelial cells, while claudin-2 and claudin-3 were undetectable [Yoshida Y., et al. Investig. Ophthalmol. Vis. Sci. 2009; 50:2103-2108; Alfuraih S., et al. Investig. Ophthalmol. Vis. Sci. 2020; 61:3]. The PDZ domain in claudins exerts its anchoring function by interacting with the intracellular ZO molecules and connecting to cytoskeletal proteins [Tsukita S., et al. Trends Biochem. Sci. 2019; 44:141-152; Gunzel D., Yu A. S. Physiol. Rev. 2013; 93:525-569]. Occludin is an integral membrane protein that contributes to tight junction stabilization, cell signaling and regulation, cell polarity, and optimal barrier function [Cummins P. M. Mol. Cell Biol. 2012; 32:242-250]. It interacts with claudins and ZO-1 to form the sealing strands of the tight junction complex. The disclosed findings demonstrating that tight junction proteins were significantly reduced under HG conditions align with prior work in the field [Jiang Q. W., et al. Acta Pharmacol. Sin. 2019; 40:1205-1211; Huang C., et al. Curr. Eye Res. 2016; 41:783-790]. Interestingly, all three treatments-combination Tβ4/VIP, and Tβ4 and VIP monotherapies-effectively prevented HG-induced decreases in ZO-1, occludin, and claudin-1 with significant increases noted with Tβ4/VIP. A previous study showed that Tβ4 significantly improved vascular permeability dysfunction and regulated tight junction protein stability [Song K., et al. Eur. J. Pharmacol. 2020; 869:172891]. Scuderi et al. reported that treatment with VIP significantly restored both claudin-1 and ZO-1 expression, and increased barrier integrity, in hyperglycemia-induced ARPE-19 cells [Scuderi S., et al. Peptides. 2013; 39:119-124]. The disclosed findings support these previous results and provide further evidence for a synergistic effect between Tβ4 and VIP in restoring tight junctions, which is thought to be a driving factor for the observed functional differences detected by ECIS.

Immunostaining confirmed the breakdown of tight junction complexes under HG conditions. All three treatment groups (Tβ4/VIP, Tβ4, VIP) maintained the expected continuous, linear pattern along cell-cell contacts, with the most intense positive staining observed in cells treated with Tβ4/VIP. While occludin is mainly localized at the tight junctions, a small fraction of occludin has been observed in intracellular compartments (endosomes, Golgi apparatus), where it is thought to regulate the assembly, trafficking, and turnover of tight junctions. The more intense intracellular occludin+ staining could also be due to endocytosis. Occludin can be internalized through clathrin-mediated or caveolae-mediated endocytosis, then transported to endosomes or lysosomes, where it can undergo degradation or recycling. Another possibility is that it may be temporarily internalized and sequestered intracellularly to allow for dynamic changes in cell shape and movement. Regardless, it is worth further investigation. It was also noticed that HG conditions altered epithelial cell shape and morphology, presenting with a more flattened or elongated appearance compared with the classic cobblestone-like morphology observed under NG conditions. This correlates with the Cm values that indicate changes in membrane structure in response to HG exposure. This morphological change was also observed in HUCLs exposed to HG but treated with Tβ4 or VIP as monotherapies, albeit less severe. Cell morphology was notably maintained in the Tβ4/VIP-treated cells despite HG exposure. Further, only with the combination treatment did the cell morphology and protein expression of ZO-1 and occludin most resemble those observed under NG conditions.

The disclosed complementary approach provides a foundation for the therapeutic effects of Tβ4/VIP regarding hyperglycemia-induced changes in corneal epithelial cell behavior, barrier integrity, and wound healing mechanisms. HG conditions have been associated with alterations in tight junction integrity and function, compromising the corneal epithelial barrier. When administered as a combination therapy, these bioactive peptides effectively mitigate the HG-induced disruptions in tight junction complexes, preserving the integrity and functionality of the corneal epithelial barrier, even during the wound healing response. However, it is prudent to acknowledge that this work was carried out using an in vitro cell culture model, which does not fully capture the complexity of in vivo conditions. As an extension of this point, the current study focuses on human corneal epithelial cells that play a major role in the structure and function of the cornea, but do not represent the entire cellular diversity and responses within the diabetic cornea. In addition, while simplified high-glucose conditions mimic hyperglycemia, the diabetic cornea is affected by multiple factors, including inflammation and oxidative stress, which are not fully replicated in the current model. That being said, future directions will include in vivo studies to validate the findings observed in vitro. Diabetic animal models will not only provide a more comprehensive understanding of the effects of Tβ4/VIP on the diabetic cornea in a physiological context, but also allow for the mechanistic exploration of the disclosed combination peptide treatment. Longitudinal studies will also provide insights into the sustained effects of Tβ4/VIP. Overall, the combined therapeutic potential of Tβ4/VIP offers a promising strategy to counteract the detrimental effects of HG on the corneal epithelium and warrants further investigation into its protective role regarding the diabetic cornea and related complications.
(vii) Closing Paragraphs. As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in an embodiment's ability to promote wound healing in a diabetic subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of promoting wound healing and/or treating an ocular condition in a subject comprising up-regulating in the subject
thymosin beta 4 (Tb4) and/or a precursor, active fragment, or active variant thereof; and
vasoactive peptide (VIP) and/or a precursor, active fragment, or active variant thereof
thereby promoting wound healing and/or treating the ocular condition in the subject.

2. The method of claim 1, wherein the up-regulating is through administering
a therapeutically effective amount of the thymosin beta 4 (Tb4) and/or precursor, active fragment, or active variant thereof and
a therapeutically effective amount of the vasoactive peptide (VIP) and/or precursor, active fragment, or active variant thereof to the subject.

3. The method of claim 1, wherein the precursor, active fragment, or active variant of Tb4 comprises SEQ ID NO: 2, 3, or 4, Tb4X, or proTb4.

4. The method of claim 1, wherein the precursor, active fragment, or active variant of VIP comprises PreproVIP, ProVIP, N-terminal VIP fragment (1-12), or C-terminal VIP fragment (22-28).

5. The method of claim 1, wherein the subject is a diabetic subject.

6. The method of claim 2, wherein the administering is systemic and/or topical.

7. The method of claim 2, wherein the administering is intradermal, intralesional, intravitreal, intraocular, and/or subcutaneous.

8. The method of claim 2, wherein the administering is through application of a wound dressing.

9. The method of claim 2, wherein the therapeutically effective amounts are administered as multiple doses.

10. The method of claim 9, wherein the multiple doses are administered every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly.

11. The method of claim 1, wherein the upregulating occurs with administration of a second wound treatment.

12. The method of claim 11, wherein the second wound treatment comprises an antiplatelet medication.

13. The method of claim 11, wherein the second wound treatment comprises an anti-infective agent.

14. The method of claim 11, wherein the second wound treatment comprises an anesthetic.

15. The method of claim 1, wherein the promoting wound healing promotes re-epithelialization.

16. The method of claim 1, wherein promoting wound healing and/or treating an ocular condition reduces or delays vision loss, drusen, pigment changes in a retina, abnormal blood vessel growth, leaky blood vessels, macular swelling, corneal swelling, corneal thinning, accumulation of a fatty yellow pigment (lipofuscin), night blindness, distorted vision, blurry vision, rod damage, cone damage, uvea inflammation, eye redness, pain, sensitivity to light, floaters, eye flashes, nodules, orbital inflammation, lacrimal gland enlargement, decreased visual acuity, decreased contrast sensitivity, blind spots, loss of color perception, loss of peripheral vision, fluid build-up in a macula, retinal scarring, double vision, pigment clumps, tunnel vision, thin cornea, spotting, leukocoria, lesions, crystals, or nystagmus,

17. The method of claim 1, wherein the ocular condition comprises keratoepitheliopathy, impaired corneal wound healing, or corneal neuropathy.

18. The method of claim 1, wherein the ocular condition comprises diabetic retinopathy, diabetic macular edema, corneal ulcer, Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Beliefs disease, Achromatopsia 2, acute posterior multifocal placoid pigment epitheliopathy (APMPPE), acute zonal occult outer retinopathy (AZOOR), adult-onset vitelliform macular dystrophy (AVMD), ocular albinism with late-onset sensorineural deafness (OASD), Alstrom syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline comeoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, or Wagner syndrome.

19. A composition comprising
thymosin beta 4 (Tb4), a precursor, active fragment, and/or active variant thereof; and
vasoactive peptide (VIP), a precursor, active fragment, and/or active variant thereof; and
a pharmaceutically acceptable carrier.

20. The composition of claim 19, wherein the precursor, active fragment, or active variant of Tb4 comprises SEQ ID NO: 2, 3, or 4, Tb4X, or proTb4.

21. The composition of claim 19, wherein the precursor, active fragment, or active variant of VIP comprises PreproVIP, ProVIP, N-terminal VIP fragment (1-12), or C-terminal VIP fragment (22-28).

22. The composition of claim 19, formulated for systemic, topical, intradermal, intralesional, intravitreal, intraocular, and/or subcutaneous administration.

23. The composition of claim 19, formulated as a gel, ointment, paste, lotion, cream, spray, foam, or powder.

24. The composition of claim 19, formulated for sustained release.

25. The composition of claim 19, incorporated into a wound dressing.

26. The composition of claim 25, wherein the wound dressing is a bandage or a transdermal patch.

27. The composition of claim 26, wherein the bandage comprises an adhesive bandages.

28. The composition of claim 27, wherein the adhesive bandage comprises a pressure sensitive adhesive.

29. A kit comprising
thymosin beta 4 (Tb4), a precursor, active fragment, and/or active variant thereof; and
vasoactive peptide (VIP), a precursor, active fragment, and/or active variant thereof.

30. The kit of claim 29, wherein the precursor, active fragment, or active variant of Tb4 comprises SEQ ID NO: 2, 3, or 4, Tb4X, or proTb4.

31. The kit of claim 29, wherein the precursor, active fragment, or active variant of VIP comprises PreproVIP, ProVIP, N-terminal VIP fragment (1-12), or C-terminal VIP fragment (22-28).

32. The kit of claim 29, further comprising a pharmaceutically acceptable carrier.

33. The kit of claim 29, further comprising a wound dressing.

34. The kit of claim 33, wherein the wound dressing is a bandage or a transdermal patch.

35. The kit of claim 34, wherein the bandage comprises an adhesive bandages.

36. The kit of claim 35, wherein the adhesive bandage comprises a pressure sensitive adhesive.

37. The kit of claim 29, further comprising a second wound treatment.

38. The kit of claim 37, wherein the second wound treatment comprises an antiplatelet medication.

39. The kit of claim 37, wherein the second wound treatment comprises an anti-infective agent.

40. The kit of claim 37, wherein the second wound treatment comprises an anesthetic.


SEQUENCE LISTING (5 sequences)

SEQ ID NO: 1 - protein, 82 aa, Homo sapiens
  SITE 1: note = Acetylation of the N terminus
SDKPDMAEIEKFDKSKLKKTETQEKNPLPSKETIEQEKQAHIDNEMEEEAVDGSNGERGARAEANKNAGPRTTGAPRPAKSA

SEQ ID NO: 2 - protein, 4 aa, Homo sapiens
  SITE 1: note = Acetylation of the N terminus
SDKP

SEQ ID NO: 3 - protein, 4 aa, Homo sapiens
SDKP

SEQ ID NO: 4 - protein, 4 aa, Homo sapiens
KPGP

SEQ ID NO: 5 - protein, 28 aa, Homo sapiens
HSDAVFTDNYTRLRKQMAVKKYLNSILN